(19) United States
(12) Patent Application Publication
(10) Pub. No.: US 2002/0147145 A1
(43) Pub. Date: Oct. 10, 2002

(54) Materials and methods relating to the degradation of Cdc25A in response to DNA damage

(75) Inventors: Niels Mailand, Kobenhavn (DK); Jacob Falck Hansen, Kobenhavn (DK); Jiri Bartek, Greve (DK); Jiri Lukas, Greve (DK); Claudia Lukas, Greve (DK); Randi Syljuasen, Kobenhavn (DK); Karsten Lundgren, Fredensborg (DK)

Correspondence Address: Dike, Bronstein, Roberts & Cushman, Intellectual Property Practice Group, EDWARDS & ANGELL, LLP, P.O. Box 9169, Boston, MA, 02209, US

(73) Assignee: Zealand Pharmaceuticals A/S

(21) Appl. No.: 09/949,196

(22) Filed: Sep. 7, 2001

Related U.S. Application Data
(63) Continuation-in-part of application No. PCT/GB01/01008, filed on Mar. 8, 2001.

(30) Foreign Application Priority Data
Mar. 8, 2000 (GB) GB 0005573.1
Jan. 15, 2001 (GB) GB 0101021.4

(51) Int. Cl. A61K 38/17; A61K 31/366; A61K 31/4745; A61N 5/00

(52) U.S. Cl. 514/12; 514/453; 514/283; 600/1

(57) ABSTRACT

Cdc25A has a role in a further signalling pathway for DNA repair which operates in response to DNA damage, in which Chk1 or Chk2 are activated following DNA damage and phosphorylate Cdc25A at one or more serine residues, and more particularly at Ser123 and/or Ser262 and/or Ser292 and/or Ser504. The phosphorylated Cdc25A is then recognized by the F-box protein and is then degraded in a proteasome dependent manner, thereby allowing the cells to undergo cell cycle arrest and repair. Accordingly, by interfering with the phosphorylation and/or degradation of Cdc25A and/or using other strategies to maintain Cdc25A level, this pathway can be used to prevent cells from undergoing repair and thereby increasing the accumulation of DNA damage in the cells, e.g. increasing the fraction of tumor cells which can be killed by DNA damaging therapeutic agents, such as radiation or anti-tumor drugs, or which undergo apoptosis.

### CROSS-REFERENCE TO RELATED APPLICATIONS

The present application is a continuation-in-part of PCT/GB01/01008 filed on Mar. 8, 2001 by N. Mailand et al. which application claims the benefit of British Application Nos. GB 0005573.1 and GB 0101021.4 filed on Mar. 8, 2000 and Jan. 15, 2001, respectively. The disclosures of the PCT/GB01/01008, GB 0005573.1 and GB 0101021.4 applications are incorporated herein by reference.

### FIELD OF THE INVENTION

The present invention relates to materials and methods relating to the degradation of cdc25A in response to DNA damage, and in particular to the uses of this response in the treatment of cancer and other hyperproliferative disorders. The present invention further relates to methods of screening for compounds capable of modulating the interaction of Cdc25A and other cell cycle proteins.

### BACKGROUND OF THE INVENTION

One of the fundamental biological processes conserved throughout evolution is the ability of all eukaryotes to secure high fidelity of the genetic transmission. Genomic stability is ensured by a plethora of cell cycle checkpoints, surveillance pathways which ultimately inhibit cyclin-dependent kinases (CDKs) and/or the anaphase-promoting complex, key enzymes that orchestrate progression through the cell cycle. Checkpoints capable of arresting mammalian cells in G1 phase, thereby preventing replication of the damaged DNA, appear to be largely dependent on the ability of the p53 tumour suppressor to induce the CDK inhibitor p21^{WAF1}/CIP1/SDI1. However, transient inhibition of Cdk2, the essential activity required for G1/S progression, has been reported also in cells with compromised function of p53, and fibroblasts isolated from mice with homozygously deleted p21 gene still respond by at least attenuated delay of G1/S transition upon DNA damage.

U.S. Pat. No. 5,756,335 (Beach & Galaktionov) discloses the cloning of the human Cdc25A and Cdc25B and identifies their activity as endogenous tyrosine phosphatases and their involvement in the cell cycle in regulating the activation of Cdc2-kinase. This patent discloses that Cdc25A and Cdc25B are part of a multigene family having endogenous tyrosine kinase activity and which activate cyclin B in the absence of Cdc2. The patent shows that Cdc25 levels do not change in Xenopus in either the meiotic maturation or early embryonic division cycles. It further shows that Cdc25 physically associates with Cdc2/cyclin B in a cell cycle dependent manner, that maximal association between Cdc25 and Cdc2/cyclin B occurs at the time of maximal kinase activity and that Cdc2 associated with Cdc25 is tyrosine dephosphorylated and active as a kinase. The patent discloses that Cdc25A is overexpressed in some forms of cancer and suggests regulating the activity of Cdc2 by controlling the interaction of Cdc25 with Cdc2, cyclin B or the Cdc2/cyclin B complex. Methods of treating hyperproliferative disorders such as cancer are suggested using anti-sense oligonucleotides to block Cdc25 production, blocking a Cdc25 transcription factor, degrading Cdc25 with a protease, using agents capable of binding to Cdc25 or cyclin B or inhibiting the activating domain of cyclin B.

WO99/11795 discloses the identification of human effector cell cycle checkpoint kinase 1 (Chk1) and that expression of Chk1 decreases the sensitivity of the cell to DNA damage. The cloning of cell cycle checkpoint kinase 2 (Chk2) is described in Matsuoka et al, 1998. Further Chk2 sequences are disclosed in Chaturvedi et al, 1999, and Blasina et al, 1999.

### SUMMARY OF THE INVENTION

Broadly, the present invention is based on the discovery of the role of Cdc25A in a further signalling pathway for DNA repair which operates in response to DNA damage, e.g. as caused by radiation or chemical agents such as anti-tumour drugs. This pathway is distinct from the p53/p21^{WAF1} pathway described in the prior art and has different characteristics. In this signalling pathway, Chk1 or Chk2 are activated following DNA damage and phosphorylate Cdc25A at one or more serine residues, and more particularly at Ser123 and/or Ser262 and/or Ser292 and/or Ser504. The phosphorylated Cdc25A is then recognised by the F-box protein part of ubiquitin ligase and is then degraded in a proteasome dependent manner, thereby allowing the cells to undergo cell cycle arrest and repair. Accordingly, by interfering with the phosphorylation and/or degradation of Cdc25A and/or using other strategies to maintain Cdc25A level, this pathway can be inhibited, preventing cells from undergoing repair and thereby increasing the accumulation of DNA damage in the cells. By directing this treatment so that the accumulation of DNA damage is favoured in populations of diseased cells, it is in turn possible to increase the fraction of such cells which can be killed by DNA damaging therapeutic agents, such as radiation or anti-tumour drugs, or which undergo apoptosis. As tumour cells are often defective in other checkpoints (e.g. many tumour cells are p53 defective) and repair pathways as compared to normal cells, this means that the approach can be used to preferentially sensitise the tumour cells to these treatments, reducing the severity of side effects associated with these treatments or increasing their effectiveness.

It is believed that the invention is better appreciated by reference to FIG. 10 (providing an overview of CDC25A Function as a Therapeutic Target).

The approach of maintaining Cdc25A in cells in the face of a degradation pathway induced by DNA damage to sensitise the cells to treatment is distinct from that disclosed in U.S. Pat. No. 5,756,335 which says that Cdc25A is present at a steady state concentration and is a positive cell cycle regulator responsible for removing the inhibitory phosphate from cell dependent kinases such as cdk2, thereby allowing cells to enter mitosis and proliferate. Thus, rather than maintaining Cdc25A levels by inhibiting its degradation in response to DNA damage, the prior art suggests inhibiting the proliferation of tumour cells by inhibiting Cdc25A or reducing its expression in those cells.

Accordingly, in a first aspect, the present invention provides the use of a substance which is capable of inhibiting the interaction of Cdc25A and Chk1 or Chk2 for the preparation of a medicament for the treatment of a cancer or a hyperproliferative disorder. The inhibition of the interaction may be the binding of Cdc25A to Chk1 or Chk2 or the inhibition of the phosphorylation of Cdc25A and thus its subsequent degradation in response to DNA damage.

In a further aspect, the present invention provides a substance having the property of binding to Chk1 or Chk2 and inhibiting the phosphorylation of Cdc25A by the Chk1 or Chk2, the substance comprising:

(a) a peptide fragment of between 5 and 30 amino acids which has at least 80% sequence identity with a corresponding sequence of Cdc25A, the fragment including a serine residue at a position corresponding to amino acid Ser123 or Ser262 or Ser292 or Ser504 in Cdc25A; or,

(b) a derivative of peptide fragment (a); or

(c) a substance which is peptide fragment (a) or derivative (b) linked to a coupling partner.

Under (b), examples of preferred derivatives of the invention include those in which the serine residue set out in (a) is substituted for a different amino acid residue as described in more detail below, or is deleted.

In further aspects, the present invention provides an isolated nucleic acid molecule encoding one of these substances, expression vectors comprising the nucleic acid and host cells transformed with the vectors. A method of producing the substance is also provided, the method comprising culturing the host cells and isolating the substance thus produced.

In a further aspect, the present invention provides the substance for use in a method of medical treatment.

In a further aspect, the present invention provides the use of the substances for identifying (i) binding partners of the substance or (ii) compounds having the property of binding to Chk1 or Chk2 and inhibiting the phosphorylation of Cdc25A.

In a further aspect, the present invention provides a method of identifying compounds capable of modulating the interaction of Cdc25A and Chk1 or Chk2, the method comprising:

(a) contacting (i) a substance comprising Cdc25A or a fragment or variant thereof, (ii) a substance comprising Chk1 or Chk2 or a fragment or variant thereof and (iii) a candidate compound, under conditions wherein, in the absence of the candidate compound, said substances interact; and,

(b) determining the interaction between said substances to identify whether the candidate compound modulates the interaction.

In a further aspect, the present invention provides a method of identifying binding partners of a substance having the property of binding to Chk1 or Chk2 and inhibiting the phosphorylation of Cdc25A by the Chk1 or Chk2, the substance comprising a peptide fragment of between 5 and 30 amino acids having at least 80% sequence identity with a corresponding sequence of Cdc25A which has a serine residue at a position corresponding to amino acid Ser123 or Ser262 or Ser292 or Ser 504 in Cdc25A, the method comprising contacting the substance and a candidate compound and determining whether the candidate compound has the property of binding to the substance.

In these method aspects of the invention, the interaction determined in step (b) can be the binding of Cdc25A to Chk1 or Chk2, the phosphorylation of Cdc25A by Chk1 or Chk2 and/or the determination of whether the compounds is capable of causing G1/S arrest in a population of cells.

In a further aspect, the present invention provides the use of an amino acid motif having between 2 and 30 amino acids from Cdc25A and having a serine at a position corresponding to Ser123 or Ser262 or Ser292 or Ser504 in full length Cdc25A in the design of an compound which is modelled to resemble the three dimensional structure, the steric size, and/or the charge distribution of said amino acid motif, or a portion thereof, wherein the compound has the property of binding to Chk1 or Chk2, and optionally being phosphorylated by the kinase.

In a further aspect, the present invention provides the use of a substance which is capable of disrupting the interaction of phosphorylated Cdc25A and a F-box protein involved in its degradation for the preparation of a medicament for the treatment of cancer or a hyperproliferative disorder, wherein the inhibition of the interaction inhibits the degradation of the Cdc25A in response to DNA damage.

In a further aspect, the present invention provides a method of identifying compounds capable of inhibiting the ubiquitination and degradation of phosphorylated Cdc25A upon binding by a F-box protein, the method comprising:

(a) contacting (i) a substance comprising Cdc25A or a fragment or variant thereof, (ii) a F-box protein or a complex including a F-box protein and (iii) a candidate compound, under conditions wherein, in the absence of the candidate compound, the F-box protein targets the Cdc25A for ubiquitination and degradation; and,

(b) determining whether the compound inhibits the degradation of the Cdc25A.

In a further aspect, the present invention provides a substance having the competing with phosphorylated Cdc25A for binding to a F-box protein, the substance comprising:

(a) a peptide fragment of between 5 and 30 amino acids which has at least 80% sequence identity with a corresponding sequence of Cdc25A, the fragment including a serine residue at a position corresponding to amino acid Ser123 or Ser262 or Ser292 or Ser504 in Cdc25A, wherein the serine residue is phosphorylated; or,

(b) a derivative of peptide fragment (a); or

(c) a substance which is peptide fragment (a) or derivative (b) linked to a coupling partner.

In a further aspect, the present invention provides the use of any of the above substances for raising antibodies, and in particular antibodies which are capable of specifically binding to Cdc25A in either a phosphorylated or unphosphorylated form.

Embodiments of the present invention will now be described by way of example with reference to the accompanying figures.

### BRIEF DESCRIPTION OF THE DRAWINGS

FIG. 1. Proteasome-dependent degradation of Cdc25A in response to DNA damage. (A) UV specifically inhibits the activity of Cdc25A, but not Cdc25B and Cdc25C. Asynchronous U-2-OS cells were treated with UV (15 J/m²), harvested 2 hours later and assayed for phosphatase activities of individual Cdc25s as described. (B) UV-irradiation downregulates the level of Cdc25A protein. Cells were treated as in (A), and expression of Cdc25s was detected by immunoblotting. (C) UV-induced downregulation of Cdc25A is prevented by the proteasome inhibitor LLnL (25 μg/ml), added to the culture medium of irradiated or mock-treated U-2-OS cells, as evidenced by immunoblotting and phosphatase activity analyses of lysates prepared 1 hour after UV-treatment. (D) Ectopic Cdc25A is degraded in a proteasome-dependent manner in UV-irradiated U-2-OS/B3C4 cells, induced to express the transgene for 12 h, followed by exposure to UV and treatment with proteasome inhibitors as in (C). Immunoblotting was performed with Cdc25A-specific antibody DCS-122. (E) Ubiquitination of Cdc25A in vivo. U-2-OS cells were co-transfected with expression plasmids for HA-tagged Cdc25A or p21 (positive control) together with a 6×His-tagged expression vector for ubiquitin. After denaturing cell lysis, the polyubiquitinated species were purified on a Ni²⁺ column and subsequently visualized by immunoblotting with anti-HA antibody as described. (F) UV-irradiation decreases the half-life of Cdc25A protein in U-2-OS/B3C4 cells, treated as in (D). Cycloheximide (25 μg/ml) was added to the medium 1 hour after irradiation and cells were harvested at the indicated times. HACdc25A was detected by immunoblotting with DCS-122 antibody. (G) UV-induced downregulation of Cdc25A is conserved in human diploid fibroblasts (IMR-90), exposed to UV (15 J/m²), and assayed by immunoblotting and phosphatase assays 2 hours after irradiation. (H) Downregulation of Cdc25A in response to γ-irradiation. U-2-OS cells were treated with 8 Gy of γ-irradiation, harvested 2 hours later, and lysates were subjected to immunoblotting and phosphatase activity analyses of Cdc25A.

FIG. 2. Rapid, UV-induced degradation of Cdc25A inhibits the activity of cyclin E-cdk2. (A) Total levels of cyclin E and Cdc25A, their associated kinase/phosphatase activities, and association of p21 and cdk2 with cyclin E in cell extracts of U-2-OS cells harvested at the indicated times after UV-irradiation. (B) The rates of tyrosine dephosphorylation and kinase reactivation of cyclin E-cdk2 are decreased by UV-treatment. U-2-OS cells were exposed to UV or left untreated and harvested 1 hour later. Dephosphorylation of cdk2 was initiated by addition of 10 mM EDTA and lysates were incubated at 30° C. At the indicated times, cyclin E immunoprecipitates were immunoblotted with specific antibodies to Tyr15-phosphorylated cdk2 or total cdk2 (left panels), and the activity of immunoprecipitated cyclin E-cdk2 measured in an in vitro kinase assay (right panel). Results are shown as the mean values and standard deviations from three independent experiments. (C) Ectopic Cdc25A expression was induced in U-2-OS/B3C4 cells by removal of tetracycline from the medium 3 hours prior to treatment. Dephosphorylation and activation of cdk2 was done as described in (B). At indicated times, cyclin E immunoprecipitates were assayed for histone H1 kinase activty. The mean values and standard deviations from three independent experiments are shown.

FIG. 3. UV-induced degradation of Cdc25A is p53-independent but requires the integrity of the Chk1 kinase. (A) Induction of dominant-negative p53 (p53DD) by removal of tetracycline for 24 hours abrogates p53-dependent transcription in U-2-OS/C6 cells, as revealed by immunostaining for luciferase upon microinjection with a p53-dependent reporter plasmid (mdm2-Luc, 25 μg/ml needle concentration) (left panel). Cdc25A abundance and activity were reduced 2 hours after UV-treatment as determined by immunoblotting and phosphatase activity analyses, regardless of repression (+tet) or induction (−tet) of p53 DD in U-2-OS/C6 cells 24 hours before irradiation (right panels). (B) The Cdc25A response is preserved in p53-negative cells. Asynchronous SAOS-2 cells were irradiated with UV (15 J/m²) or left untreated. Cells were harvested 2 hours later and lysates were subjected to immunoblotting and phosphatase activity measurements. (C) UV stimulates association of Cdc25A and Chk1 in U-2-OS cells transiently transfected with HA-tagged Cdc25A expression plasmid and either mock-treated, UV-irradiated (15 J/m²) or UV-irradiated after a 1 hour preincubation with caffeine (10 mM). After an additional 2 hours, cells were lysed and analyzed for levels and complexes of HACdc25A and Chk1 as indicated. (D) Caffeine prevents UV-mediated downregulation of Cdc25A. U-2-OS cells were treated with UV and caffeine as in (C), and Cdc25A protein level and phosphatase activity were analyzed as described. (E) The activity of Chk1 towards Cdc25A is increased by UV. Chk1 was immunoprecipitated from lysates of U-2-OS cells treated as in (B), and its activity determined in an in vitro kinase assay in the presence or absence of the Chk1 inhibitor, UCN-01 (1 μM), using purified GST-Cdc25A as a substrate. (F) UV-mediated degradation of Cdc25A is prevented by the specific Chk1 inhibitor, UCN-01. Cells were treated as in (D), except that caffeine was substituted with 300 μM UCN-01, and Cdc25A protein level and activity were assayed as described.

FIG. 4. UV irradiation inhibits DNA replication. (A) Asynchronously growing U-2-OS cells were irradiated with UV-C (15 J/m²), two hours later pulse-labelled for 10 min with BrdU (100 μg/ml), and analyzed by multivariate flow cytometry (FL1-H, the BrdU signal revealed by the FITC-conjugated anti-BrdU antibody; FL2-A, the DNA content obtained by counterstaining the cells with propidium iodide). The numbers indicate the relative amount of cells actively synthesizing DNA. (B) U-2-OS cells were arrested in M phase by 12 hour incubation in the presence of nocodazole. Eight hours after release, the cells were exposed to UV light as described in (A) and incubated for another 6 hours before staining with propidium iodide and analyzing for DNA distribution by flow cytometry. The numbers indicate the relative amount of cells that entered S phase. (C) Sustained level of Cdc25A increases the amount of UV-induced DNA single strand breaks (SSB). Cdc25A expression was induced in U-2-OS/B3C4 cells by removal of tetracycline from the medium 3 hours prior to treatment. Cells were irradiated with the indicated doses of UV and incubated 30 minutes DNA single strand breaks were measured by alkaline elution. (D) Colony formation assay performed in U-2-OS/B3C4 cells exposed to the indicated doses of UV light. Cdc25A protein was transiently induced (−TET) as described in (C). (E) Model of the G1/S checkpoints activated by UV-induced DNA damage in human cells. The more acute response, independent on p53, involves ubiquitin/proteasome-dependent destruction of Cdc25A, triggered through phosphorylation of Cdc25A by the Chk1 kinase, and ultimately resulting in a transient G1 arrest due to inhibition of kinase activity associated with cyclin-cdk2 complexes. This is followed by activation of the classical p53/p21 pathway, which ensures more sustained cell cycle arrest. The two checkpoint responses appear sequential and thus complementary, but they may also represent a surveillance backup response for cells which have lost one of the pathways, though deregulation of either mechanism would predispose to genetic instability and development of cancer.

FIG. 5. Effects and regulation of IR-induced destruction of Cdc25A. a, DNA synthesis and Cdc25A abundance (insert) in gamma-irradiated U-2-OS/B3C4 cells repressed (+Tet) or induced (−Tet) 1 h prior IR to express ectopic HA-Cdc25A. b, Kinetics of IR-induced changes in activities and abundance of Cdc25A and Chk2, cyclin E-associated H1 kinase activity and Cdk2 Tyr-15 phosphorylation in U-2-OS cells exposed to 10 Gy. c DNA synthesis in CD20-sorted U-2-OS/T-Rex cells expressing Cdc25A and/or Cdk2AF.

FIG. 6. Failure of Chk2 mutants to interact with and induce degradation of Cdc25A results in S-phase checkpoint defect. a, Immunoblots of Chk1 and Chk2 and corresponding kinase activities towards GST-Cdc25A in untreated (−) or 10 Gy g-irradiated (+) U-2-OS cells. b, U-2-OS/B3C4 cells were transfected with Myc-Chk2 plasmids as indicated, stimulated to express HA-Cdc25A for 3h, g-irradiated (10 Gy; +) or left untreated (−), and immunoblotted for Myc-Chk2 and Cdc25A (top; WCE, whole cell extract). Anti-Myc immunoprecipitates were assayed for co-precipitated Cdc25A and for in-vitro kinase activity towards GST-Cdc25A (bottom). Asterisk, a 3-fold excess of Myc-Chk2-R145W was transfected. c and d, Various U-2-OS/Myc-Chk2 clones were analysed for Cdc25A abundance (c) and Cdk2-associated kinase activity (d) at the indicated time points after g-irradiation (10 Gy). e, DNA synthesis in various U-2-OS/Myc-Chk2 clones measured after g-irradiation (10 Gy). Open squares, U-2-OS/B3C4 cells with induced Cdc25A.

FIG. 7. IR-induced destruction of Cdc25A requires functional ATM and Chk2. a, Abundance and activities of Cdc25A and Chk2 in untreated (−) or g-irradiated (10 Gy; +) SW620 and HCT-15 cells. b, HCT-15-derived cells were g-irradiated (10 Gy; +) or left untreated (−) and analysed for activities and abundance of Cdc25A and Chk2, and cyclin E-associated H1 kinase activity and Cdk2 levels (Tyr15 phosphorylated and total). c, DNA synthesis in control vector versus Chk2-reconsitituted (Myc-Chk2) HCT-15 cells measured after g-irradiation (10 Gy). d, Normal or A-T lymphoblasts were treated and analysed as in (b).

FIG. 8. Chk2 phosphorylation of Cdc25A serine 123 triggers IR-induced destruction of Cdc25A. a, Schematic representation of human Cdc25A and the GST-coupled fragments tested for Chk2 phosphorylation. b, GST-Cdc25A fragments were incubated with wild type (WT) or catalytically inactive (KD) versions of purified GST-Chk2. Proteins were resolved by SDS-PAGE and visualized by autoradiography (top) or Coomassie blue staining (bottom). c, Amino acid sequence of the region surrounding serine 123 (underlined) in human, mouse, and rat Cdc25A, aligned with the known Chk1/2-phosphorylated region of Cdc25C. d, GST-Cdc25A(101-140) (wt) or GST-Cdc25A(101-140)(S123A) was incubated with Chk2 immunoprecipitates from untreated (−) or 10 Gy g-irradiated (+) U-2-OS cells. Proteins were resolved and visualized as in (b). e, U-2-OS cells transiently transfected with wild type or mutated (S123A) HA-Cdc25A were g-irradiated (10 Gy; +) or left untreated (−) in the presence of LLnL to prevent destruction. Less protein from S123A expressing cells was loaded for better visualization of the IR-induced slower migrating form of Cdc25A stabilised by LLnL (indicated by arrow). f, U-2-OS cells were transiently transfected with wild type or mutated (S123A) HA-Cdc25A. Twelve hours after transfection, cells were g-irradiated (10 Gy; +) or left untreated (−) prior to addition of cycloheximid (CHX; 25 μg/ml) and immunoblotted for HA-Cdc25A at the indicated time points after CHX addition. g, Schematic model of the IR-induced S-phase checkpoint pathway in normal (left) versus checkpoint-deficient (right) cells; pathway components targeted in cancer are marked by asterisk.

FIG. 9. This figure shows initial results of the in vitro kinase assay described below.

FIG. 10. This figure provides a summary of CDC25A Function as a Therapeutic Target.

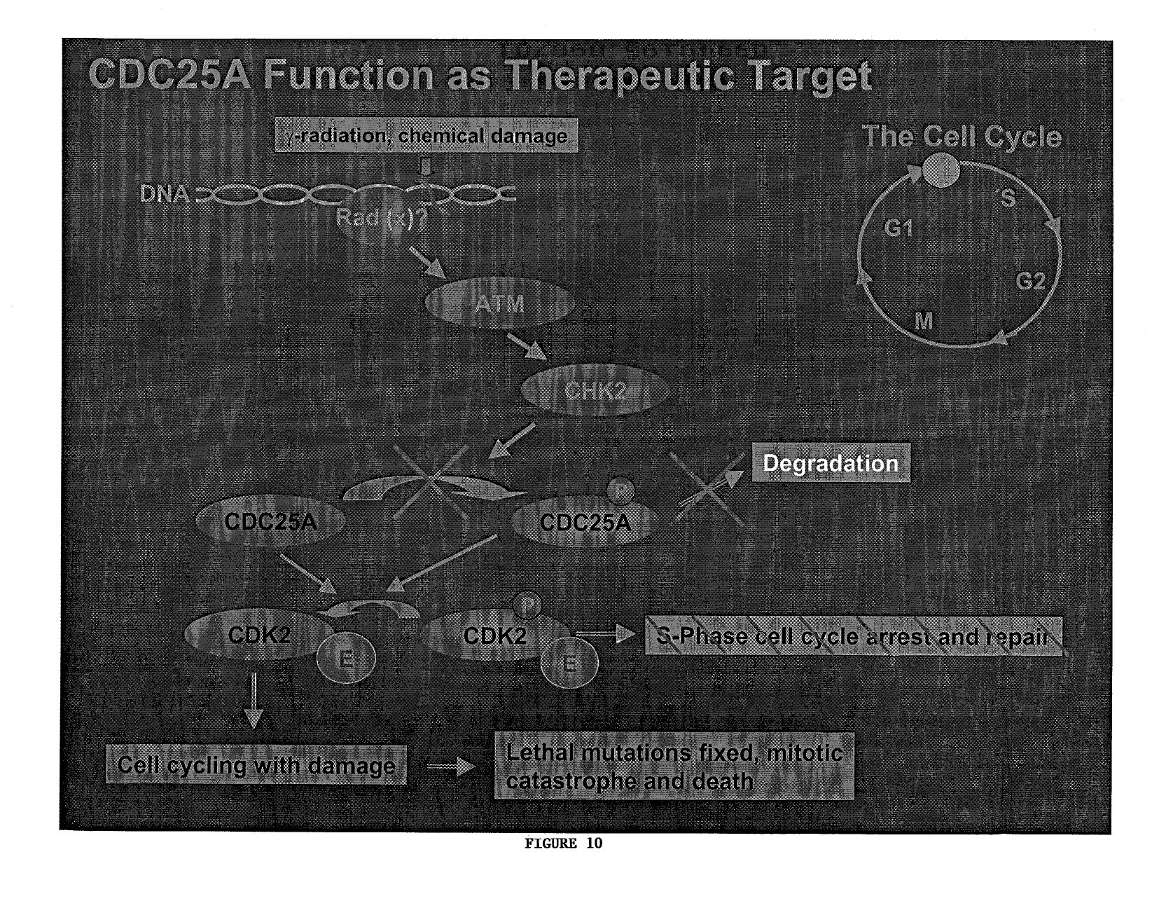

### DETAILED DESCRIPTION

The Cdc25A degradation pathway in response to DNA damage operates to transiently delay cell cycle progression in both G1 phase and inside S phase, to provide time for DNA repair and to increase cell survival. The fact that interference with the degradation of Cdc25A after DNA damage (by allowing the Cdc25A to stay active even after radiation), leads to enhanced DNA damage and cell death in human tumour cells has been reported. Importantly, the Cdc25A pathway doesn't directly induce apoptosis (unlike the p53 pathway), so in the case of the Cdc25A pathway it would be beneficial to block or prevent the pathway in order to presensitize cancer cells to therapy, whereas in case of p53, the trend is to find drugs to enhance its pro-apoptotic activity. Our Cdc25A pathway therefore belongs to the more classical checkpoint designed to allow repair and survival. It is believed that interference with this type of pathway eg., with drugs, will presensitize cells to radiation and chemotherapy.

### The Cell Cycle

The cell cycle checkpoint pathways collectively represent an important part of cell cycle control which do not interfere with normal cell proliferation, but rather monitor the progression through the cell cycle in terms of the quality of DNA, precision of DNA replication and chromosome segregation. In other words, checkpoints are a quality and fidelity control, monitoring the performance of the basic cell cycle machinery with the option to stop the cell cycle in the event DNA becomes damaged, DNA replication machinery makes errors or chromosomes are not ready to be separated properly. The cell cycle checkpoint mechanisms which are activated in response to DNA damage therefore provide time for DNA repair, and sometimes also help activate repair, allowing cells to survive such damage (if the damage is reparable), or to prevent cell cycle progression in cells with unrepaired DNA or abnormal chromosomes.

In one aspect, the approach described herein using the Chk/Cdc25A/ubiquitin pathway is based on the fact that defects or prevention of the cell cycle checkpoint functions result in the accumulation of unrepaired genetic damage or mutations, and that cells in which one or more checkpoints do not function properly are generally more sensitive to DNA damage and die more readily when exposed to radiation or other genotoxic agents than normal cells. Unrepaired or excessive DNA damage eventually triggers the cell suicide mechanisms of apoptosis or mitotic catastrophe, thus eliminating the potentially dangerous, genetically highly unstable cells. Probably the most significant difference between normal and tumour cells is that most normal cells are non proliferating, and even those which do proliferate harbour multiple functional checkpoint pathways which can respond to DNA damage to minimize genetic destabilisation (mutations). In contrast, tumour cells proliferate, lack one or several checkpoint control pathways and sometimes are also defective in the DNA repair, all features which make them more susceptible to DNA damage. In retrospect, it appears that many anti-cancer therapeutics originally found empirically, such as γ-radiation and cytotoxic drugs, operate via inducing DNA damage and that these agents are successful in view of the differences between normal and cancer cells mentioned above.

The issue of selectivity for tumours relies, at least in part, on the fact that most tumours lack some checkpoints already, unlike normal cells which can use the whole spectrum of checkpoints to reversibly block the cell cycle, and later recover. Tumour cells, meanwhile, might for instance keep the Cdc25A pathway as the only (or one of two, perhaps) remaining active checkpoint, and its neutralization by our future strategy would deprive tumour cells even of this remaining safeguard mechanism against excessive DNA damage and cell death. The present disclosure reports the molecular basis of two important checkpoints: the rapid wave of the G1 checkpoint, and the pathway based on the mechanistic knowledge of the cascade. There have been some reports concerning existence of the p53-independent branch of the G1 checkpoint, and of the intra-S-phase checkpoint. However, molecular basis for those checkpoints and use of that knowledge to achieve treatment methods is disclosed, for the first time, in the present application.

Thus, one strategy employed in the present invention is to interfere with a function of a particular checkpoint mechanism to treat cancer or a hyperproliferative disorder in which the checkpoint pathway is operational, while simultaneously or sequentially treating a patient with conventional chemotherapy or radiotherapy. The aim of this strategy is to deprive the diseased cells of the possibility to implement the checkpoint and so deny the cells the time for proper repair. Deprived of this possibility, the affected cells shift the balance of their decisions-reactions towards irreversible arrest or cell death. The induction of cell death is more likely to occur in tumour cells than in normal cells, since the latter have multiple checkpoints operational, have more efficient repair, or proliferate less, and as a result are much more likely to survive and properly repair the effects of the treatment. By way of example an intervention of this type is shown in Chan et al, Nature 401:616-620, 1999. The pathway described in this paper is p53-dependent and it operates in G2 phase, preventing premature entry of cells into mitosis when DNA is still damaged. When the authors eliminated the critical component of this G2 checkpoint pathway downstream of p53, in this case via gene knockout, the specific 14-3-3 family member, the cells exposed to DNA damage were unable to enter a sustained G2 arrest, entered mitosis prematurely, and massively died due to a mitotic catastrophe.

The novel checkpoint mechanism disclosed herein in which the rapid phosphorylation and degradation of Cdc25A blocks cells at G1/S and in S phase belongs to the ‘classical’ reversible category. Thus, the degradation and/or inactivation of Cdc25A following DNA damage is rapid and only temporary (imposed within 30-60 minutes and lasting for some 3-4 hours when rather low doses of UV or other DNA damaging agents are used), and the majority of the cells do recover from this transient arrest and continue proliferating. The reversible nature and impact of this pathway on cell survival is shown in experiments described herein in which control U-2-OS cells are compared with U-2-OS cells in which the level of the Cdc25A protein is elevated to a level which cannot be efficiently degraded by the cellular machinery activated by the DNA damage, with the result that the cells are unable to implement this response. The results showed that the clonogenic growth, a generally accepted parameter reflecting cell survival after DNA damage, was significantly lower in cells with the experimentally impaired Cdc25A checkpoint response. In related experiment, the extent of DNA damage accumulating after UV-irradiation was measured, and again the amount of DNA single strand breaks was considerably more apparent in the cells with elevated Cdc25A, i.e. those cells which can not degrade the Cdc25A protein and thus implement the checkpoint response properly. These results are consistent with the concept that this pathway is a bona fide checkpoint response, and further shows that interfering with the pathway can significantly affect accumulation of DNA damage and survival of cells exposed to DNA damaging agents.

These findings suggest a different strategy to U.S. Pat. No. 5,441,880 to exploit the role of Cdc25A in the cell cycle which does not rely on simply blocking the activity or production of Cdc25A phosphatase and so inhibit cells entering mitosis and thereby proliferating. This difference is based on the distinct role of Cdc25A in cell cycle control:

(1) Cdc25A is required as a positive cell cycle regulator in all somatic cycling cells, due to its ability to remove the inhibitory phosphate from cyclin-dependent kinases such as cdk2, the partner of cyclin E and cyclin A at the G1/S transition and in S phase.

(2) The second role of Cdc25A which is the subject of the present invention, namely that Cdc25A serves as a target of the novel checkpoint mechanism activated in response to DNA damage in which the Cdc25A protein becomes phosphorylated and degraded, thereby preventing its essential positive role for G1/S and resulting in cell cycle arrest. This pathway is only operational when cells are exposed to DNA damage.

Thus, in some aspects, the present invention concerns interrupting the signalling pathway to Cdc25A after DNA damage or interfering with execution of this pathway human tumour cells, thereby increasing the accumulation of DNA damage and the fraction of cells normally killed by the DNA damaging therapeutics such as radiation and anti-tumour drugs. The aim of this strategy is therefore to sensitize tumour cells towards the action of the DNA-damaging cancer treatments with the scope to either achieve the same treatment effects with lower doses of radiation/drugs, thereby decreasing some of the adverse side effects of the existing therapies, or achieving more pronounced elimination of diseased cells.

In one embodiment which is discussed further below, this strategy employs peptides which can interfere with the key signalling events in the Cdc25A pathway, namely phosphorylation of the Cdc25A protein by the upstream kinases(s) activated upon DNA damage such as Chk1 or Chk2 and the interaction of the phosphorylated Cdc25A with the F-box protein, which is part of ubiquitin ligase which triggers ubiquitination and degradation of Cdc25A. The results disclosed herein show that Chk1 is involved in this process upon UV radiation and it is known Chk2 has a similar role in response to gamma radiation.

The residue of Cdc25A targeted for phosphorylation by Chk1 or Chk2 appears to be a serine residues and more particularly Ser123 or Ser262 or Ser292 or Ser504 of the human Cdc25A, as deduced from phosphopeptide mapping of Cdc25A isolated from cells after DNA damage, and confirmed by site-directed mutagenesis. The phosphosite corresponding to Ser292 is a dominant one upon response to both UV or gamma radiation, i.e. the site is shared by Chk1 and Chk2. Thus, when disrupting peptides are delivered into cells, either by microinjection, electroporation or by use of a coupling partner such as a transport molecule which allow the peptides to cross cell membranes, just before or concomitant with DNA damage insults such as radiation, they can compete for binding to activated Chk1 or Chk2 kinases in the cells, and thereby inhibit or even prevent the interaction of the kinase with the endogenous Cdc25A, in turn resulting in the presence of an amount of Cdc25A sufficient to inhibit or prevent the cells employing the checkpoint.

In an alternative aspect, the downstream recognition of the phosphorylated Cdc25A protein by a protein ubiquitination enzyme can be targeted to inhibit or prevent it leading to reduction of Cdc25A levels in response to DNA damage. The ubiquitination enzyme is most likely via a member of the so-called ‘F-box protein’ family of proteins which are components of ubiquitin ligases which recognize the specific phosphorylated residue in proteins to be targeted for ubiquitination and degradation, in the present case marking Cdc25A for degradation by the proteasome machinery. The experiments below show that Cdc25A is turned over by the ubiquitin-dependent, proteasome-mediated degradation.

Further experiments described herein examine the activation of checkpoint pathways in eukaryotic cells when exposed to ionizing radiation (IR) in order to delay cell cycle progression. Defects in the IR-induced S-phase checkpoint cause radioresistant DNA synthesis (RDS), a phenomenon identified in cancer-prone ataxia-telangiectasia (A-T) patients suffering mutations in the ATM gene. The work disclosed herein identifies an interplay between ATM, Chk2 kinase and Cdc25A phosphatase, and shows that deregulation of this mechanism leads to RDS. Cdc25A activates cyclin dependent kinase 2 (Cdk2) required for DNA replication, and it becomes degraded in response to DNA damage. IR-induced destruction of Cdc25A required ATM, and Chk2-mediated phosphorylation on serine 123. The resulting loss of Cdc25A protein precluded activating dephosphorylation of Cdk2 and led to transient DNA replication blockade. Tumour-associated Chk2 alleles were unable to bind and phosphorylate Cdc25A. Cells expressing these Chk2 alleles, elevated Cdc25A, or a Cdk2 mutant unable to undergo inhibitory phosphorylation (Cdk2AF) failed to inhibit DNA synthesis when irradiated. These results highlight serine 123 phosphorylation as a crucial step in IR-induced Cdc25A degradation, support Chk2 as a candidate tumour suppressor, and identify the ATM-Chk2-Cdc25A-Cdk2 pathway as a genomic integrity checkpoint, which prevents RDS.

Peptides based on the sequences identified by this work for modulating the interaction of Cdc25A and Chk1 and/or Chk2 may further be refined in sequence or these interactions can be used to screen for other selective inhibitors. These substances can then be used in combination with existing chemotherapy or radiotherapy as discussed in more detail below.

The advantage of covering both the conditional checkpoint/target function and the constitutive proliferation-associated function of Cdc25A via proteolysis machinery would be that using either strategies might be application to all human malignancies and by extension other hyperproliferative disorders. Overexpression of Cdc25A is a common feature of human cancers. Gasparotto et al. ((1997) Cancer Res. 57: 2366-68) found overexpression of Cdc25A in 70% of tumors of head and breast Cancers; Wu et al. ((1998) Cancer Res. 58: 4082-85) found a 60% overexpression of Cdc25A in primary non-small cell lung cancer with high correlation to poor differentiation; and it has been shown that Cdc25A is overexpressed in 47% of breast cancer tumors correlating with high levels of Cdk2 activity (J. Clin. Invest. (2000) 106: 753-61). Since more than 50% of human cancers have mutations in p53 (Greenblatt et al. (1994) Cancer Res. 54: 4855-78; Carson and Lois (1995) Lancet 346: 1009-11) it will appear that the Cdc25A pathway is a very promising target for cancer therapy.

### Peptides and Peptidomimetics

One class of substances that can be used to inhibit the interaction or binding of Cdc25A and either (a) Chk1 or Chk2 or (b) a F-box protein which forms part of aa ubiquitin ligases are peptides based on the sequence motifs of Cdc25A or these proteins, and variants or derivatives of the peptides.

The wild type Cdc25A amino acid and nucleic acid sequences are disclosed in U.S. Pat. No. 5,441,880 and WO93/10242. The wild type Chk1 amino acid and nucleic acid sequences are disclosed in WO99/11795. The wild type Chk2 amino acid and nucleic acid sequences are disclosed in Matsuoka et al, 1998, Chaturvedi et al, 1999, and Blasina et al, 1999.

The sequence annotation for the amino acid residues of Cdc25A used herein are in accordance with the annotation used in U.S. Pat. No. 5,441,880. However, the NCBI protein database accession XP_{—}037169 submitted Aug. 23, 2001 has a slightly different sequence of the first 13 amino acid residues of the human Cdc25A resulting in the serine residues 123, 262, 292 and 504 being annotated as serine 124, 263, 293 and 505.

Preferably, the peptide is based on the Cdc25A sequence and includes the phosphorylation site targeted by Chk1 or Chk2, namely Ser292 of wild type human Cdc25A, e.g. comprising a motif of 2, 3, 4, 5, 6, 7, 8, 10, 20 or 30 amino acids from the sequence below and including a serine residue in a position corresponding to Ser123 or Ser262 or Ser292 or Ser504 of Cdc25A, optionally in combination with one or more amino acid alterations as discussed below. The sequence surrounding the Ser292 phosphorylation site in human Cdc25A is as follows:

Gly Ser Thr Lys Arg Arg Lys Ser Met Ser Gly Ala Ser Pro Lys Glu
285 290 295 300
The sequence surrounding the Ser123 phosphorylation site in
human Cdc25A is as follows:
Ser Pro Ala Leu Lys Arg Ser His Ser Asp Ser Leu Asp His Asp Ile Phe
115 120 125 130
The sequence surrounding the Ser504 in human Cdc25A is as follows:
Glu Asp Leu Lys Lys Phe Arg Thr Lys Ser Arg Thr Trp Ala Gly Glu Lys
495 500 505 500
The sequence surrounding the Ser262 in human Cdc25A is as follows:
Cys Lys Leu Phe Asp Ser Pro Ser Leu Cys Ser Ser Ser Thr Arg Ser Val
255 260 265 270

In other embodiments, the present invention provides variant peptides based on this motif of Cdc25A in which Ser123 or Ser 262 or Ser292 or Ser504 is substituted by a residue such as alanine which is non-phosphorlyatable or resists phosphorylation, or by a residue such glutamic acid which mimics phosphorylation.

In other embodiments, the present invention provides variant peptides in which the serine residue is phosphorylated. These peptides may be used along with any of the peptides disclosed herein to immunize mice and rabbits to prepare antibodies specifically recognizing the Cdc25A, and more especially antibodies which are capable of specifically binding to Cdc25A which has been phosphorylated after DNA damage by Chk2/Chk1 or which is unphosphorylated. The phosphorlyated Cdc25A peptides, and especially those phosphorylated at Ser123, can also be used, possibly in a cell permeable form, to try to block the pathway downstream of Cdc25A, by preventing the recognition of the phosphorylated Cdc25A by the proteolytic machinery, and in particular the F-box protein which recognizes the sequence around the phosphoserine 123 to put the ubiquitin chain on Cdc25A to mark it for destruction by the proteasome. This method could be employed alone or in combination with other approaches described herein to block the pathway between Chk2 and Cdc25A, and between Cdc25A and the destruction machinery.

In general, peptides of the present invention are small molecules, and are preferably less than 40 amino acids in length, more preferably less than 35 amino acids, more preferably less than 30 amino acids, more preferably less than 25 amino acids, more preferably less than 20 amino acids, more preferably less than 15 amino acids, more preferably less than 10 amino acids, or 9, 8, 7, 6, 5, 4 amino acids in length. The present invention also encompasses peptides which are sequence variants or derivatives of a wild type Cdc25A, Chk1 or Chk2 or F-box sequences.

As is well understood, identity at the amino acid level is generally defined and determined by the TBLASTN program, of Altschul et al, J. Mol. Biol., 215:403-10, 1990, which is in standard use in the art. Sequence identity may be over the full-length of the relevant peptide or over a contiguous sequence of about 5, 10, 15, 20, 25, 30 or 35 amino acids, compared with the relevant wild-type amino acid sequence. Preferably, the amino acid sequence of the peptides of the invention share at least 75%, or 80%, or 85% identity, and more preferably at least 90% or 95% identity sequence identity with the corresponding part of the full length human Cdc25A, Chk1, Chk2 or F-box sequences.

The present invention also provides sequence variants of the above peptides. In one embodiment, the variants are peptide fragments of Cdc25A including 1, 2, 3, 4, 5, greater than 5, or greater than 10 amino acid alterations such as substitutions, deletions or insertions with respect to the wild-type sequence.

Peptide derivatives of the peptides and sequence variants described above include pharmaceutically acceptable salts of the peptides, alkyl esters, amides, alkylamides, dialkylamides, wherein the alkyl groups are preferably lower alkyl such as C1-4.

The present invention further includes provides peptides which are composed of D and L amino acids, or combinations thereof. Alternatively or additionally, the peptides, variants and derivatives may be part of a larger peptide, which may or may not include an additional portion of Cdc25A, e.g. 1, 2, 3, 4, 5 or 10 or more additional amino acids, adjacent to the relevant specific peptide fragment in Cdc25A, or heterologous thereto may be included at one end or both ends of the peptide.

### Coupling Partners

The invention also includes derivatives of the peptides, including the peptide linked to a coupling partner, e.g. an effector molecule, an immunogen, a label, a drug, a toxin and/or a carrier or transport molecule. Techniques for coupling the peptides of the invention to both peptidyl and non-peptidyl coupling partners are well known in the art. In one embodiment, the carrier molecule is a 16 aa peptide sequence derived from the homeodomain of Antennapedia (e.g. as sold under the name “Penetratin”), which can be coupled to a peptide via a terminal Cys residue. The “Penetratin” molecule and its properties are described in WO91/18981.

### Synthesis

Peptides may be generated wholly or partly by chemical synthesis. The compounds of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, N.Y. (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

### Expression

Another convenient way of producing a peptidyl molecule according to the present invention (peptide or polypeptide) is to express nucleic acid encoding it, by use of nucleic acid in an expression system. Accordingly the present invention also provides in various aspects nucleic acid encoding the polypeptides and peptides of the invention.

Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression.

Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding a polypeptide or peptide in accordance with the present invention can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992), given the nucleic acid sequence and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding p21 fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the Cdc25A sequences can be made, e.g. using site directed mutagenesis, to lead to the expression of modified Cdc25A peptide or to take account of codon preference in the host cells used to express the nucleic acid.

In order to obtain expression of the nucleic acid sequences, the sequences can be incorporated in a vector having one or more control sequences operably linked to the nucleic acid to control its expression. The vectors may include other sequences such as promoters or enhancers to drive the expression of the inserted nucleic acid, nucleic acid sequences so that the polypeptide or peptide is produced as a fusion and/or nucleic acid encoding secretion signals so that the polypeptide produced in the host cell is secreted from the cell. Polypeptide can then be obtained by transforming the vectors into host cells in which the vector is functional, culturing the host cells so that the polypeptide is produced and recovering the polypeptide from the host cells or the surrounding medium. Prokaryotic and eukaryotic cells are used for this purpose in the art, including strains of E. coli, yeast, and eukaryotic cells such as COS or CHO cells.

Accordingly, the present invention also encompasses a method of making a polypeptide or peptide, the method including expression from nucleic acid encoding the polypeptide or peptide. This may conveniently be achieved by growing a host cell in culture, containing such a vector, under appropriate conditions which cause or allow expression of the polypeptide. Polypeptides and peptides may also be expressed in in vitro systems, such as reticulocyte lysate.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells, U-2-OS cells, SAOS-2 cells and many others. A common, preferred bacterial host is E. coli.

Thus, a further aspect of the present invention provides a host cell containing heterologous nucleic acid as disclosed herein.

The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell, or otherwise identifiably heterologous or foreign to the cell.

A still further aspect provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide (or peptide) is produced. If the polypeptide is expressed coupled to an appropriate signal leader peptide it may be secreted from the cell into the culture medium. Following production by expression, a polypeptide or peptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers.

Introduction of nucleic acid encoding a peptidyl molecule according to the present invention may take place in vivo by way of gene therapy, to disrupt or interfere with interaction between Cdc25A and Chk1 or Chk2

Thus, a host cell containing nucleic acid according to the present invention, e.g. as a result of introduction of the nucleic acid into the cell or into an ancestor of the cell and/or genetic alteration of the sequence endogenous to the cell or ancestor (which introduction or alteration may take place in vivo or ex vivo), may be comprised (e.g. in the soma) within an organism which is an animal, particularly a mammal, which may be human or non-human, such as rabbit, guinea pig, rat, mouse or other rodent, cat, dog, pig, sheep, goat, cattle or horse, or which is a bird, such as a chicken. Genetically modified or transgenic animals or birds comprising such a cell are also provided as further aspects of the present invention.

This may have a therapeutic aim. Also, the presence of a mutant, allele, derivative or variant sequence within cells of an organism, particularly when in place of a homologous endogenous sequence, may allow the organism to be used as a model in testing and/or studying substances which modulate activity of the encoded polypeptide in vitro or are otherwise indicated to be of therapeutic potential. Conveniently, however, assays for such substances may be carried out in vitro, within host cells or in cell-free systems.

Suitable screening methods are conventional in the art. They include techniques such as radioimmunosassay, scintillation proximetry assay and ELISA methods. Suitably either the Cdc25A protein or Chk1 or Chk2, or a fragment, an analogue, derivative, variant or functional mimetic of any of these protein, is immobilised whereupon the other is applied in the presence of the agents under test. In a scintillation proximetry assay a biotinylated protein fragment is bound to streptavidin coated scintillant—impregnated beads (produced by Amersham). Binding of radiolabelled peptide is then measured by determination of radioactivity induced scintillation as the radioactive peptide binds to the immobilized fragment. Agents which intercept this are thus inhibitors of the interaction.

### Assays

In one general aspect, the present invention provides an use of the interaction of Cdc25A and Chk1 or Chk2 for screening for substances which are capable of modulating the interaction of Cdc25A and Chk1 or Chk2. This may involve using the substances described above identifying (i) binding partners of the substance or (ii) compounds having the property of binding to Chk1 or Chk2 and inhibiting the phosphorylation of Cdc25A.

In one preferred embodiment, the present invention provides a method of identifying compounds capable of modulating the interaction of Cdc25A and Chk1 or Chk2, the method comprising:

(a) contacting (i) a substance comprising Cdc25A or a fragment or variant thereof, (ii) a substance comprising Chk1 or Chk2 or a fragment or variant thereof and (iii) a candidate compound, under conditions wherein, in the absence of the candidate compound, said substances interact; and,

(b) determining the interaction between said substances to identify whether the candidate compound modulates the interaction.

In a further aspect, the present invention provides a method of identifying binding partners of a substance having the property of binding to Chk1 or Chk2 and inhibiting the phosphorylation of Cdc25A by the Chk1 or Chk2, the substance comprising a peptide fragment of between 5 and 30 amino acids having at least 80% sequence identity with a corresponding sequence of Cdc25A, the fragment including serine at a position corresponding to amino acid Ser 123 or Ser262 or Ser292 or Ser504 in Cdc25A, the method comprising contacting the substance and a candidate compound and determining whether the candidate compound has the property of binding to the substance. In further embodiments of the invention, this method may employ sequence variants of the above mentioned Cdc25A sequences, or fragments thereof, e.g. peptides in which Ser123 or Ser262 or Ser292 or Ser504 is substituted by a residue such as alanine which is non-phosphorlyatable or resists phosphorylation, or by a residue such glutamic acid which mimics phosphorylation.

### Synthesis of Peptides

Peptide synthesis of H-Tyr-Gly-Arg-Lys-Lys-Arg-Arg-Gln-Arg-Arg-Arg-Gly-Gly-Arg-Ser-Pro-Ala-Met-Pro-NH2 (KB6) on TentaGel-S-Ram; Rapp polymere, Germany Dry TentaGel-S-Ram (0.23 mmol/g, 1 g) was placed in a polyethylene vessel equipped with a polypropylene filter for filtration and treated as described under “batchwise peptide synthesis on TentaGel resin” until finishing the coupling of the N-terminal Tyrosine. All couplings were continued over night. The acylations were checked by the ninhydrin test performed at 80° C. as earlier described. After deprotection of the Fmoc group the N-terminal amino group the peptide-resin was washed with DMF (3×15 ml, 1 min each), DCM (3×15 ml, 1 min each), diethyl ether (3×15 ml, 1 min each) and dried in vacuo.

The peptide was cleaved from the resin as described above freeze and dried from acetic acid. The identity of the peptide was confirmed by ES-MS (found MH⁺ 2440.25, calculated MH⁺ 2440.37). After purification using preparative HPLC as described above, 150 mg peptide product was collected with a purity better than 98%.

Peptide synthesis of H-Tyr-Gly-Arg-Lys-Lys-Arg-Arg-Gln-Arg-Arg-Arg-Leu-Tyr-Arg-Ser-Pro-Ala-Met-Pro-Glu-Asn-Leu-NH2 (KB5) on TentaGel-S-Ram; Rapp polymere, Germany

Dry TentaGel-S-Ram (0.23 mmol/g, 1 g) was placed in a polyethylene vessel equipped with a polypropylene filter for filtration and treated as described under “batchwise peptide synthesis on TentaGel resin” until finishing the coupling of the N-terminal Tyrosine. All couplings were continued over night. The acylations were checked by the ninhydrin test performed at 80° C. as earlier described. After deprotection of the Fmoc group the N-terminal amino group the peptide-resin was washed with DMF (3×15 ml, 1 min each), DCM (3×15 ml, 1 min each), diethyl ether (3×15 ml, 1 min each) and dried in vacuo.

The peptide was cleaved from the resin as described above freeze and dried from acetic acid. The identity of the peptide was confirmed by ES-MS (found MH⁺2829.50, calculated MH⁺ 2829.60). After purification using preparative HPLC as described above, 220 mg peptide product was collected with a purity better than 93%.

Peptide synthesis of H-Tyr-Gly-Arg-Lys-Lys-Arg-Arg-Gln-Arg-Arg-Arg-Thr-Lys-Arg-Arg-Lys-Ala-Met-Ser-Gly-Ala-Ser-NH2 (KB7) on TentaGel-S-Ram; Rapp polymere, Germany

Dry TentaGel-S-Ram (0.23 mmol/g, 1 g) was placed in a polyethylene vessel equipped with a polypropylene filter for filtration and treated as described under “batchwise peptide synthesis on TentaGel resin” until finishing the coupling of the N-terminal Tyrosine. All couplings were continued over night. The acylations were checked by the ninhydrin test performed at 80° C. as earlier described. After deprotection of the Fmoc group the N-terminal amino group the peptide-resin was washed with DMF (3×15 ml, 1 min each), DCM (3×15 ml, 1 min each), diethyl ether (3×15 ml, 1 min each) and dried in vacuo.

The peptide was cleaved from the resin as described above freeze and dried from acetic acid. The identity of the peptide was confirmed by ES-MS (found MH⁺2731.5, calculated MH⁺ 2731.61). After purification using preparative HPLC as described above, 97 mg peptide product was collected with a purity better than 90%.

Peptide synthesis of H-Tyr-Gly-Arg-Lys-Lys-Arg-Arg-Gln-Arg-Arg-Arg-Leu-Phe-Asp-Ser-Pro-Ala-Leu-Cys-Ser-Ser-Ser-NH2 (KB8) on TentaGel-S-Ram; Rapp polymere, Germany

Dry TentaGel-S-Ram (0.23 mmol/g, 1 g) was placed in a polyethylene vessel equipped with a polypropylene filter for filtration and treated as described under “batchwise peptide synthesis on TentaGel resin” until finishing the coupling of the N-terminal Tyrosine. All couplings were continued over night. The acylations were checked by the ninhydrin test performed at 80° C. as earlier described. After deprotection of the Fmoc group the N-terminal amino group the peptide-resin was washed with DMF (3×15 ml, 1 min each), DCM (3×15 ml, 1 min each), diethyl ether (3×15 ml, 1 min each) and dried in vacuo.

The peptide was cleaved from the resin as described above freeze and dried from acetic acid. The identity of the peptide was confirmed by ES-MS (found MH⁺2665.38, calculated MH⁺ 2565.46). After purification using preparative HPLC as described above, 118 mg peptide product was collected with a purity better than 96%.

Peptide synthesis of H-Tyr-Gly-Arg-Lys-Lys-Arg-Arg-Gln-Arg-Arg-Arg-Leu-Lys-Arg-Ser-His-Ser-Asp-Ser-Leu-Asp-His-NH2 (KB10) on TentaGel-S-Ram; Rapp polymere, Germany

Dry TentaGel-S-Ram (0.23 mmol/g, 1 g) was placed in a polyethylene vessel equipped with a polypropylene filter for filtration and treated as described under “batchwise peptide synthesis on TentaGel resin” until finishing the coupling of the N-terminal Tyrosine. All couplings were continued over night. The acylations were checked by the ninhydrin test performed at 80° C. as earlier described. After deprotection of the Fmoc group the N-terminal amino group the peptide-resin was washed with DMF (3×15 ml, 1 min each), DCM (3×15 ml, 1 min each), diethyl ether (3×15 ml, 1 min each) and dried in vacuo.

The peptide was cleaved from the resin as described above freeze and dried from acetic acid. The identity of the peptide was confirmed by ES-MS (found MH⁺2833.25, calculated MH⁺ 2833.60). After purification using preparative HPLC as described above, 70 mg peptide product was collected with a purity better than 98%.

Peptide synthesis of H-Tyr-Gly-Arg-Lys-Lys-Arg-Arg-Gln-Arg-Arg-Arg-Leu-Lys-Arg-Ser-His-Ala-Asp-Ser-Leu-Asp-His-NH2 (KB11) on TentaGel-S-Ram; Rapp polymere, Germany

Dry TentaGel-S-Ram (0.23 mmol/g, 1 g) was placed in a polyethylene vessel equipped with a polypropylene filter for filtration and treated as described under “batchwise peptide synthesis on TentaGel resin” until finishing the coupling of the N-terminal Tyrosine. All couplings were continued over night. The acylations were checked by the ninhydrin test performed at 80° C. as earlier described. After deprotection of the Fmoc group the N-terminal amino group the peptide-resin was washed with DMF (3×15 ml, 1 min each), DCM (3×15 ml, 1 min each), diethyl ether (3×15 ml, 1 min each) and dried in vacuo.

The peptide was cleaved from the resin as described above freeze and dried from acetic acid. The identity of the peptide was confirmed by ES-MS (found MH⁺2817.50, calculated MH⁺ 2817.60). After purification using preparative HPLC as described above, 70 mg peptide product was collected with a purity better than 96%.

KB10 ia a 22 amino acid peptide fragment of Cdc25A comprising the serine 123 residue and KB11 is the 22 amino acid peptide fragment of Cdc25A having an S123A substitution. These peptides are useful in screening for a substance able to bind Cdc25A and/or having the activity of inhibiting the phosphorylation of Cdc25A at Ser123

All of the above peptides were synthesized batchwise in a polyethylene vessel equipped with a polypropylene filter for filtration using 9-fluorenylmethyloxycarbonyl (Fmoc) as N-α-amino protecting group and suitable common protection groups for side-chain functionalities and using the following reagents and experimental conditions.

### Solvents

Solvent DMF (N,N-dimethylformamide, Riedel de-Häen, Germany) was purified by passing through a column packed with a strong cation exchange resin (Lewatit S 100 MB/H strong acid, Bayer AG Leverkusen, Germany) and analyzed for free amines prior to use by addition of 3,4-dihydro-3-hydroxy-4-oxo-1,2,3-benzotriazine (Dhbt-OH) giving rise to a yellow color (Dhbt-O- anion) if free amines are present. Solvent DCM (dichloromethane, analytical grade, Riedel de-Häen, Germany) was used directly without purification. Acetonitril (HPLC-grade, Lab-Scan, Dublin Ireland) was used directly without purification.

### Amino acids

Fmoc-protected amino acids were purchased from Advanced ChemTech (ACT) in suitabel side-chain protected forms.

### Coupling Reagents

Coupling reagent diisopropylcarbodiimide (DIC) was purchased from Riedel de-Häen, Germany.

### Solid Supports

Peptides were synthesized on TentaGel S resins 0.22-0.31 mmol/g. TentaGel S-Ram, TentaGel S RAM-Lys(Boc)Fmoc (Rapp polymere, Germany) were used in cases where a C-terminal amidated peptide was preferred, while TentaGel S PHB, TentaGel S PHB Lys(Boc)Fmoc were used when a C-terminal free carboxylic acid was preferred.

### Catalysts and Other Reagents

Diisopropylethylamine (DIEA) was purchased from Aldrich, Germany, piperidine and pyridine from Riedel-de Häen, Frankfurt, Germany. Ethandithiol was purchased from Riedel-de Häen, Frankfurt, Germany. 3,4-dihydro-3-hydroxy-4-oxo-1,2,3-benzotriazine (Dhbt-OH), 1-hydroxybenzotriazole (HOBt) (HOAt) were obtained from Fluka, Switzerland. Acetic anhydride was obtained from Fluka.

### Coupling Procedures

The amino acids were coupled as in situ generated HObt or HOAt esters made from appropriate N-α-protected amino acids and HObt or HOAt by means of DIC in DMF. Acylations were checked by the ninhydrin test performed at 80° C. in order to prevent Fmoc deprotection during the test (Larsen, B. D. and Holm, A., Int. J. Peptide Protein Res. 43, 1994, 1-9).

### Deprotection of the N-α-amino Protecting Group (Fmoc)

Deprotection of the Fmoc group was performed by treatment with 20% piperidine in DMF (1×5 and 1×10 min.), followed by wash with DMF (5×15 ml, 5 min. each) until no yellow color could be detected after addition of Dhbt-OH to the drained DMF.

### Coupling of HOBt-esters

3 eq. N-α-amino protected amino acid was dissolved in DMF together with 3 eq. HObt and 3 eq DIC and then added to the resin.

### Acetylation of the N-terminal Amino Group With Acetic Anhydride

40 eq acetic anhydride was dissolved in DMF together with 5 eq pyridine and then added to the resin. The acylation was checked by the ninhydrin test as described above.

### Trifluoroacetylation of the N-terminal Amino Group With Ethyl Trifluoroacetate

30 eq ethyl trifluoroacetate was dissolved in dichloromethane together with 10 eq triethyl amine and then added to resin. The acylation was checked by ninhydrin test as described above.

### Cleavage of Peptide From Resin With Acid

Peptides were cleaved from the resins by treatment with 95% triflouroacetic acid (TFA, Riedel-de Häen, Frankfurt, Germany)-water v/v or with 95% TFA and 5% ethandithiol v/v at r.t. for 2 h. The filtered resins were washed with 95% TFA-water and filtrates and washings evaporated under reduced pressure. The residue was washed with ether and freeze dried from acetic acid-water. The crude freeze dried product was analyzed by high-performance liquid chromatography (HPLC) and identified by mass spectrometry (MS).

### Batchwise Peptide Synthesis on TentaGel Resin (PEG-PS)

TentaGel resin (1 g, 0.23-0.24 mmol/g) was placed in a polyethylene vessel equipped with a polypropylene filter for filtration. The resin was swelled in DMF (15ml), and treated with 20% piperidine in DMF in order to remove the initial Fmoc group either on the linker TentaGel S RAM or on the first amino acid on the resin TentaGel S RAM-Lys(Boc)Fmoc. The resin was drained and washed with DMF until no yellow color could be detected after addition of Dhbt-OH to the drained DMF. The amino acids according to the sequence were coupled as preformed Fmoc-protected HObt esters (3 eq.) as described above. The couplings were continued for 2 h, unless otherwise specified. The resin was drained and washed with DMF (5×15 ml, 5 min each) in order to remove excess reagent. All acylations were checked by the ninhydrin test as described above. After completed synthesis the peptide-resin was washed with DMF (3×15 ml, 5 min each), DCM (3×15 ml, 1 min each) and finally diethyl ether (3×15 ml, 1 min each) and dried in vacuo. The peptide was cleaved from the resin as described earlier and the crude peptide product was analysed and purified as described below

### HPLC Conditions

Gradient HPLC analysis was done using a Hewlett Packard HP 1100 HPLC system consisting of a HP 1100 Quaternary Pump, a HP 1100 Autosampler a HP 1100 Column Thermostat and HP 1100 Multiple Wavelength Detector. Hewlett Packard Chemstation for LC software (rev. A.06.01) was used for instrument control and data acquisition. The following columns and HPLC buffer system was used:

Column: VYDAC 238TP5415, C-18, 5 mm, 300 Å 150×4.6 mm.

Buffers: A: 0.1% TFA in MQV; B: 0.085% TFA, 10% MQV, 90% MeCN.

Gradient: 0-1.5 min. 0% B

1.5-25 min 50% B

25-30 min 100% B

30-35 min 100% B

35-40 min 0% B

Flow 1, ml/min, oven temperature 40° C., UV detection: 1=215 nm. HPLC purification of the crude peptide

The crude peptide products were purified PerSeptive Biosystems VISION Workstation. VISION 3.0 software was used for instrument control and data acquisition. The following column and HPLC buffer system was used:

Column: Kromasil KR 100 Å, 10 mm C-8, 250×50.8 mm.

Buffer system: Buffers: A: 0.1% TFA in MQV; B: 0.085% TFA, 10% MQV, 90% MeCN.

Gradient: 0-37 min. 0-40% B

Flow 35 ml/min, UV detection: 1=215 nm and 280 nm.

### Mass Spectroscopy

The peptides were dissolved in super gradient methanol (Labscan, Dublin, Ireland), milli-Q water (Millipore, Bedford, Mass.) and formic acid (Merck, Damstadt, Germany) (50:50:0.1 v/v/v) to give concentrations between 1 and 10 mg/ml. The peptide solutions (20 ml) were analysed in positive polarity mode by ESI-TOF-MS using a LCT mass spectrometer (Micromass, Manchester, UK).

### Phosphorylated Peptides for Raising Antibodies

The following peptide were synthesised using standard solid phase methods of peptide synthesis using the Fmoc strategy.

H-CGCSPALKRSHS S¹²³ (H₂PO₄)DSLDHDIFQL-OH was used for raising phosphospecific antibodies against serine 123 of Cdc25A as described herein and H-CGCSPALKRSHS¹²³DSLDHDIFQL-OH was used to raise antibodies useful as negative controls.

H-CKGDLKKFRTK S⁵⁰⁴ (H₂PO₄)TRWAGEKSKR-OH can be used for raising phosphospecific antibodies against serine 504 of Cdc25A, and H-CKGDLKKFRTKS⁵⁰⁴TRWAGEKSKR-OH can be used to raise antibodies useful as negative controls.

The peptide fragments of the invention may be linked by any convenient covalent bond, preferably to the N-terminus, to a coupling partner consisting preferably of a small peptide sequence to form a peptide conjugate. The term “peptide conjugate” as used herein indicates a fusion between at least two peptide sequences via a peptidic bond or an equivalent bioisosteric bond, such as the peptide bond mimetics described in Table 1 in Tayar et al., Amino Acids (1995) 8:125-139. Said coupling partner is preferably selected from the group consisting of an HIV tat peptide residues 49-57, HIV tat peptide residues 49-56, the tat sequence YGRKKRRQRRR, a polyarginine peptide having from 6 to 20 residues, such as R₆ , and transducing peptide sequences which are able to maintain sufficient levels of peptide conjugate within cells and which does not interfere with the folding of the peptide fragment, such as the following peptide sequences: YARKARRQARR, YARAAARQARA, YARAARRAARR, YARAARRAARA, ARRRRRRRRR, and YAAARRRRRRR, which are disclosed in WO 99/29721 and in U.S. Pat. No. 6,221,355 (seq. id. nos. 3-8) the disclosures of which are incorporated herein by reference.

The peptide fragments used herein or useful in the methods described herein consist preferably of a sequence of about 11 amino acid residues corresponding to a sequence of Cdc25A around one of the serine 123, serine 262, serine 292 or serine 504 residues positioned in the middle of the sequence. The serine residue may be substituted with a serine analogue, i.e. an amino acid having properties similar to serine, or, preferably resembling serine but being unphosphorylatable. Examples of serine analogues are alanine, L-Abu (L-2-aminobutanoic acid), Aib (2-aminoisobutanoic acid), beta-alanine, Aoa (aminooxyacetic acid), Val, Leu, L-Nva (L-2-aminovaleric acid), Sar (sarcosine) and Ile. An advantage of substituting serine with an unnatural amino acid residue is a possible protection of the peptide fragment against proteolytic degradation. Amidation of the C-terminus also reduces susceptibility to proteolytic degradation and is preferred in the peptide fragments of the invention.

Examples of peptide conjugates comprising a fragment of Cdc25A as defined herein are:

YGRKKRRQRRR-LEDSPALCSSS-NH2 (KB8(25A-S262A)),

YGRKKRRQRRR-TKRRKAMSGAS-NH2 (KB7(25A-S292A)),

YGRKKRRQRRR-KFRTKATRWAG-NH2,

YGRKKRRQRRR-LKRSHADSLDH-NH2,

YARKARRQARR-LFDSPALCSSS-NH2,

YARKARRQARR-TKRRKAMSGAS-NH2,

YARKARRQARR-KFRTKLTRWAG-NH2,

YARKARRQARR-LKRSHLDSLDH-NH2,

YARAARRAARR-LFDSPALCSSS-NH2,

YARAARRAARR-TKRRKAMSGAS-NH2,

YARAARRAARR-KFRTKLTRWAG-NH2,

YARAARRAARR-LKRSHLDSLDH-NH2,

YGRKKRRQRRR-LFDSPSLCSSS-NH2,

YGRKKRRQRRR-TKRRKSMSGAS-NH2,

YGRKKRRQRRR-KFRTKSTRWAG-NH2,

YGRKKRRQRRR-LKRSHSDSLDH-NH2,

YARKARRQARR-LFDSPSLCSSS-NH2,

YARKARRQARR-TKRRKSMSGAS-NH2,

YARKARRQARR-KFRTKSTRWAG-NH2,

YARKARRQARR-LKRSHSDSLDH-NH2,

YARAARRAARR-LFDSPSLCSSS-NH2,

YARAARRAARR-TKRRKSMSGAS-NH2,

YARAARRAARR-KFRTKSTRWAG-NH2; AND

YARAARRAARR-LKRSHSDSLDH-NH2.

In both of these methods, modulation of the Cdc25A with Chk1 or Chk2 interaction by a candidate compound can be assessed by determining the presence or extent of the binding or disruption of Cdc25A to Chk1 or Chk2, by determining the presence or extent of the phosphorylation of Cdc25A by Chk1 or Chk2, or by determining the presence or amount of Cdc25A present in a cell based assay. These determinations can be combined with the determination of whether the candidate compound is capable of causing G1/S arrest in a population of cells, e.g. in cell based assay.

Performance of an assay method according to the present invention may be followed by isolation and/or manufacture and/or use of a compound, substance or molecule which tests positive for ability to interfere with interaction between Cdc25A and Chk1 or Chk2 and/or inhibit the phosphorylation of Cdc25A by Chk1 or Chk2.

In carrying out these methods, it may be convenient to screen a plurality of candidate compounds, e.g. as present in a library, at the same time, e.g. by contacting a mixture of different candidate compounds with the interacting peptides, and then in the event of a positive result resolving which member of the mixture is active. These technique are used in high throughput screening (HTS) to increase the numbers of compounds, e.g. resulting from combinatorial chemistry program or present in library derived from a natural source material, which can be screened in a method.

In a further aspect, the present invention provides a method of identifying compounds capable of inhibiting the degradation of phosphorylated Cdc25A by a F-box protein, the method comprising:

(a) contacting (i) a substance comprising Cdc25A or a fragment or variant thereof, (ii) a F-box protein or a complex including a F-box protein and (iii) a candidate compound, under conditions wherein, in the absence of the candidate compound, the F-box protein degrades the Cdc25A; and,

(b) determining whether the compound inhibits the degradation of the Cdc25A.

The precise format of the assays of the invention may be varied by those of skill in the art using routine skill and knowledge. For example, interaction between substances may be studied in vitro by labelling one with a detectable label and bringing it into contact with the other which has been immobilised on a solid support. Suitable detectable labels, especially for peptidyl substances include ³⁵S-methionine which may be incorporated into recombinantly produced peptides and polypeptides. Recombinantly produced peptides and polypeptides may also be expressed as a fusion protein containing an epitope which can be labelled with an antibody. Fusions can also be used to display the peptide fragments of Cdc25A, Chk1, Chk2 or F-box protein as an inserted motif, e.g. in a protein such as thioredoxin, in order to present the peptide motifs in a correct three dimensional structure.

The protein which is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

In one embodiment, the screening method looks for small molecules in the chemical compound libraries, which would inhibit phosphorylation of the Cdc25A fragment, e.g. a GST-fusion protein with the fragment of Cdc25A amino acids 100-140 or an analogous peptide. The assay detects the extent to which the candidate compounds modulate (e.g. inhibit) the phosphorlyation of the amino acid corresponding to Ser123 in full length Cdc25A. In one preferred format, the assay employs a solid phase such as a plastic well plate, on which the GST-Cdc25A fragment can be immobilised at one or more locations. The Cdc25A can then be exposed Chk1 or Chk2 which would normally phosphorylate the Cdc25A peptide. This can be readily detected either by incorporation of radioactive phosphate into the peptide and counting radioactivity, or more preferably by using a phosphospecific antibody against Ser123, produced as using one of the peptides of the invention as discussed above. The detection step can be carried out after washing off the Chk from the plate and the use of the antibody has the advantage of being very specific and non-radioactive. Thus, the screening method may include one or more of the following steps:

(a) immobilising the Cdc25A peptide on the solid support;

(b) bringing the Chk 1 or 2 and one or more candidate compounds into contact with the solid support under conditions in which the Cdc25A peptide, the Chk 1 or 2 and the candidate compound can interact;

(c) washing the solid support to remove the Chk1 o2 and the candidate compounds;

(d) detecting the extent to which the candidate compounds modulate the phosphorylation of Cdc25A by the Chk1 or 2; and,

(e) selecting one or mor candidate compounds which modulate the phosphorylation of Cdc25A by Chk1 or 2.

The inhibitory compounds identified using the assay of the invention might be expected to fall into at least two possible categories: direct inhibitors of Chk2, or those which interfere with the recognition and/or phosphorylation of Cdc25A by Chk2. Both types of compounds might be useful for the present invention and might be subject to further characterisation.

An assay according to the present invention may also take the form of an in vivo assay. The in vivo assay may be performed in a cell line such as a yeast strain or mammalian cell line in which the relevant polypeptides or peptides are expressed from one or more vectors introduced into the cell.

The amount of candidate substance or compound which may be added to an assay of the invention will normally be determined by trial and error depending upon the type of compound used. Typically, from about 0.01 to 100 nM concentrations of putative inhibitor compound may be used, for example from 0.1 to 10 nM. Greater concentrations may be used when a peptide is the test substance.

Compounds which may be used may be natural or synthetic chemical compounds used in drug screening programmes. Extracts of plants which contain several characterised or uncharacterised components may also be used.

### Antibodies

Antibodies directed to the site of interaction in either protein form a further class of putative inhibitor compounds. As mentioned above, the present invention further provides the use of the peptides and substances of the present invention for raising antibodies and in particular for raising antibodies which are capable of recognising Cdc25A phosphorylation sites in either a phosphorylated or dephosphorylated form. Candidate inhibitor antibodies may be characterised and their binding regions determined to provide single chain antibodies and fragments thereof which are responsible for disrupting the interaction.

Antibodies may be obtained using techniques which are standard in the art. Methods of producing antibodies include immunising a mammal with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature 357:80-82, 1992). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding substance having a binding domain with the required specificity. Thus, the invention covers antibody fragments, derivatives, functional equivalents and homologues of antibodies, including synthetic molecules and molecules whose shape mimics that of an antibody enabling it to bind an antigen or epitope.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The mode of determining binding is not a feature of the present invention and those skilled in the art are able to choose a suitable mode according to their preference and general knowledge.

Antibodies may also be used in purifying and/or isolating a polypeptide or peptide according to the present invention, for instance following production of the polypeptide or peptide by expression from encoding nucleic acid therefor. Antibodies may be useful in a therapeutic context (which may include prophylaxis) to disrupt Cdc25, Chk1 or Chk2 interaction with a view to inhibiting Cdc25A phosphorylation. Antibodies can for instance be microinjected into cells.

### Mimetic Compounds

Other candidate inhibitor compounds may be based on modelling the 3-dimensional structure of a polypeptide or peptide fragment and using rational drug design to provide potential inhibitor compounds with particular molecular shape, size and charge characteristics.

Following identification of a substance or agent which modulates or affects the phosphorylation of Cdc25A by Chk1 or Chk2, the substance or agent may be investigated further.

As noted, the agent may be peptidyl, e.g. a peptide which includes a sequence as recited above, or may be a functional analogue of such a peptide.

As used herein, the expression “functional analogue” relates to peptide variants or organic compounds having the same functional activity as the peptide in question, which may interfere with the binding between Cdc25A and Chk1 or Chk2. Examples of such analogues include chemical compounds which are modelled to resemble the three dimensional structure of the Cdc25A or Chk1 or Chk2 domain in the contact area, and in particular the arrangement of the key amino acid residues identified above as they appear in human Cdc25A.

Accordingly, the present invention provides a method of designing mimetic compounds having the property of inhibiting the phosphorylation of Cdc25A by Chk1 or Chk2 said method comprising:

(i) analysing a substance having the biological activity to determine the amino acid residues essential and important for the activity to define a pharmacophore; and,

(ii) modelling the pharmacophore to design and/or screen candidate mimetics having the biological activity.

In a further aspect, the present invention provides the use of an amino acid motif having between 2 and 30 amino acids from Cdc25A and having a serine at a position corresponding to Ser123 and/or Ser262 and/or Ser292 and/or Ser504 in full length Cdc25A in the design of an compound which is modelled to resemble the three dimensional structure, the steric size, and/or the charge distribution of said amino acid motif, the wherein the compound has the property of binding to Chk1 or Chk2.

Suitable modelling techniques are known in the art. This includes the design of so-called “mimetics” which involves the study of the functional interactions fluorogenic oligonucleotide the molecules and the design of compounds which contain functional groups arranged in such a manner that they could reproduced those interactions.

The designing of mimetics to a known pharmaceutically active compound is a known approach to the development of pharmaceuticals based on a lead compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for further testing or optimisation, e.g. in vivo or clinical testing.

The mimetic or mimetics found by this approach can then be screened to see whether they have the target property, or to what extent they exhibit it. Further optimisation or modification can then be carried out to arrive at one or more final mimetics for in vivo or clinical testing.

Mimetics of this type together with their use in therapy form a further aspect of the invention.

The present invention further provides the use of a peptide which includes a sequence as disclosed, or a derivative, active portion, analogue, variant or mimetic, thereof able to bind Chk1 or Chk2, in screening for a substance able to bind Cdc25A and/or having the activity of inhibiting the phosphorylation of Cdc25A, e.g. at Ser123 and/or Ser262 and/or Ser292 and/or Ser504.

### Pharmaceutical Uses

The substances of the invention can be used in the treatment cancer and other hyperproliferative disorders such as psoriasis, arteriogenesis or inflammation, and in particular in the treatment of conditions in which the inhibition the degradation of Cdc25A in response to DNA damage can be employed to sensitised diseased cells to a further treatment such as chemotherapy or radiotherapy.

As discussed above, this inhibition might be achieved by inhibiting the binding of Cdc25A to Chk1 or Chk2, the inhibition of the phosphorylation of Cdc25A by these kinases and/or the inhibition of the binding of phosphorylated Cdc25A to the F-box proteins which recognise and degrade it. In general, the aim of the combination of the chemotherapy or radiotherapy and the substances and uses of the present invention is to reduce the amount or frequency of the therapies, many of which have serious side effects for a patient, or to enhance the effectiveness of a given therapy, e.g. in the proportion of diseased cells in a population which are killed or commit to apoptosis as a result of the treatment.

Examples of chemotherapeutic agents which can be employed in combination with the substances of the invention include DNA topoisomerase inhibitors, e.g. DNA topoisomerase I toxins such as Camptothecin and derivatives such as topotecan; DNA topoisomerase II toxins such as anthracyclines such as Daunorubicin, Doxorubicin and Adriamycin and epipodophyllotoxines such as etoposide and teniposide. Examples of radiotherapy include treatment with γ-radiation and X-rays.

Generally, a substance according to the present invention is provided in an isolated and/or purified form. This may include being in a composition where it represents at least about 90% active ingredient, more preferably at least about 95%, more preferably at least about 98%. Such a composition may, however, include inert carrier materials or other pharmaceutically and physiologicaly acceptable excipients. As noted below, a composition according to the present invention can include in addition to an inhibitor compound as disclosed, one or more other molecules of therapeutic use, such as an anti-tumour agent.

The present invention extends in various aspects not only to a substance identified as a modulator of Cdc25A and Chk1 or Chk2 interaction or activity, property or pathway in accordance with what is disclosed herein, but also a pharmaceutical composition, medicament, drug or other composition comprising such a substance, a method comprising administration of such a composition to a patient, e.g. for anti-cancer, use of such a substance in manufacture of a composition for administration, e.g. for the treatment of cancer, and a method of making a pharmaceutical composition comprising admixing such a substance with a pharmaceutically acceptable excipient, vehicle or carrier, and optionally other ingredients.

A substance according to the present invention such as an inhibitor of Cdc25A and Chk1 or Chk2 interaction or binding may be provided for use in a method of treatment.

The invention further provides a method of modulating Chk1 or Chk2 activity, or other Cdc25A-mediated activity in a cell, which includes administering an agent which inhibits or blocks the binding of Cdc25A to Chk1 or Chk2 protein, such a method being useful in treatment of cancer and other hyperproliferative disorders.

The invention further provides a method of treating cancer or a hyperproliferative disorder which includes administering to a patient an agent which interferes with the binding of Cdc25A to Chk1 or Chk2, or the binding of phosphorylated Cdc25A to F-box proteins. In the first instance, the substance inhibits the phosphorylation of Cdc25A by Chk1 or Chk2 and in the latter case the binding of Cdc25A to the F-box proteins which are part of ubiquitin ligase, thereby leading to its degradation.

Whether it is a polypeptide, antibody, peptide, nucleic acid molecule, small molecule, mimetic or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practioners and other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

The agent may be administered in a localised manner to a tumour site or other desired site or may be delivered in a manner in which it targets tumour or other cells.

Targeting therapies may be used to deliver the active agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering these agents directly, they may be produced in the target cells by expression from an encoding gene introduced into the cells, e.g. in a viral vector (a variant of the VDEPT technique—see below). The vector may targeted to the specific cells to be treated, or it may contain regulatory elements which are switched on more or less selectively by the target cells.

The agent may be administered in a precursor form, for conversion to the active form by an activating agent produced in, or targeted to, the cells to be treated. This type of approach is sometimes known as ADEPT or VDEPT, the former involving targeting the activating agent to the cells by conjugation to a cell-specific antibody, while the latter involves producing the activating agent, e.g. an enzyme, in a vector by expression from encoding DNA in a viral vector (see for example, EP 0 415 731 A and WO90/07936).

A composition may be administered alone or in combination with other treatments, either simultaneously or sequentially dependent upon the condition to be treated, such as cancer, virus infection or any other condition in which a Cdc25A mediated effect is desirable.

Nucleic acid according to the present invention, encoding a polypeptide or peptide able to interfere with Cdc25A and Chk1 or Chk2 interaction or binding or other Cdc25A-mediated cellular pathway or function, may be used in methods of gene therapy, for instance in treatment of individuals with the aim of preventing or curing (wholly or partially) cancer.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted tumour cells, providing long lasting effect, or alternatively the treatment may have to be repeated periodically.

A variety of vectors, both viral vectors and plasmid vectors, are known in the art, see U.S. Pat. No. 5,252,479 and WO93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

A polypeptide, peptide or other substance able to interfere with the interaction of the relevant polypeptide, peptide or other substance as disclosed herein, or a nucleic acid molecule encoding a peptidyl such molecule, may be provided in a kit, e.g. sealed in a suitable container which protects its contents from the external environment. Such a kit may include instructions for use.

### Materials and Methods

Plasmids: cDNA for Cdc25A was subloned into the BamH1 site of a pCMV^{neo} Bam expression plasmid and linked in frame at its N-terminus with the sequence coding for the haemagglutinin (HA) tag. The resulting HA-Cdc25A was than further subcloned by PCR into a pBI vector (Clontech) allowing conditional, tetracycline-regulated expression. Other plasmids and DNA used were a pCW7 expression plasmid for 6×His-tagged ubiquitin, a Mdm2/Luc reporter plasmid and cDNA for p53DD subcloned into a pBI tetracycline-regulated expression vector.

To construct the Myc-tagged Chk2 expression vector, the human Chk2 cDNA was amplified by PCR with Pfu polymerase (Stratagene) and cloned into a pcDNA3 vector (Invitrogen) containing a Myc tag. The catalytically inactive D347A, and tumour-associated R145W, and I157T mutants of Chk2 as well as the Cdc25A-S123A mutant were generated using the QuikChange Site-Directed Mutagenesis kit (Stratagene). GST-Chk2, and GST-Cdc25A full length and fragments were expressed and purified as described herein and according to standard procedures.

Cell culture: U-20S (#ATTC HTB 96) and Saos-2 (#ATTCHTB 85) are human osteogenic sarcoma cell lines. U-2-OS sublines conditionally expressing HA-Cdc25A (B3C4) or p53DD (C6C6) respectively were generated by transfection with the respective transgenes in pBI plasmids. Conditions for isolating the clones, subsequent culturing and the transgene induction were described. Human diploid fibroblast strain, IMR90 (#ATTC CCL186) was purchased from ATCC. UV irradiation was performed in a UV Stratalinker 1800 (Stratagene); the ionizing radiation was delivered by X-ray generator (RT100, Philips Medico). Caffeine and nocodazole were purchased from Sigma, specific Chk1 kinase inhibitor, UCN-01 was a gift from R. J. Schultz (Drug Synthesis & Chemistry Branch, NCI).

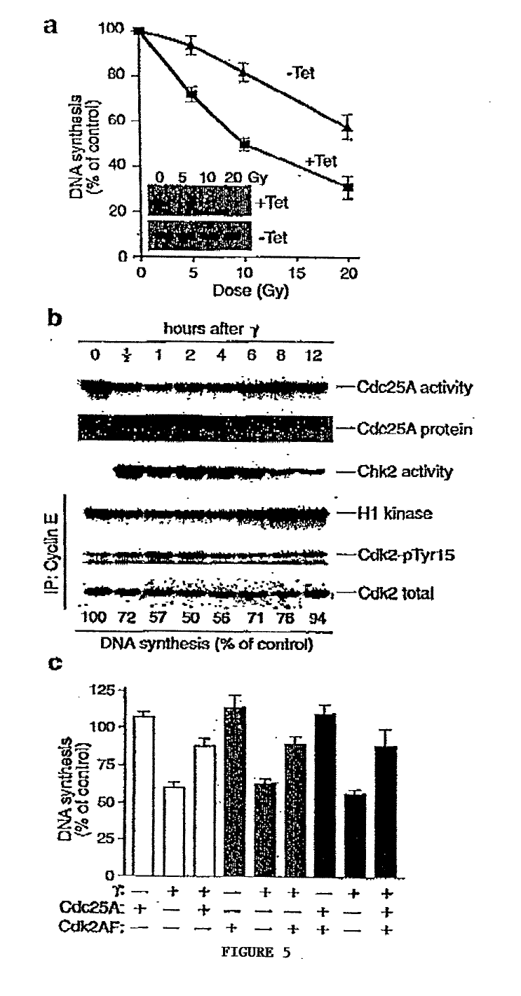

U-2-OS and SW-620 cells were grown in DMEM containing 10% foetal bovine serum. HCT-15 cells and lymphoblasts derived from normal and A-T patients were grown in RPMI containing 10% foetal bovine serum. Expression of the transgene was induced by culturing the cells in tetracycline-free growth medium for the time periods specified in figure legends. The U-2-OS-derived T-Rex cell line was purchased from Invitrogen. Expression of Cdc25A and Cdk2AF was induced by addition of tetracycline (1 μg/ml) 4 hours prior to irradiation (FIG. 5c) whereas ectopic CD20 was expressed throughout the experiment. Isolation of CD20-positive U-2-OS/T-Rex cells with anti-CD20 coupled Dynabeads was described in Santoni-Rugiu et al (Mol. Cell. Biol. 20, 3497-3509 (2000)). The U-2-OS- and HCT-15 derived polyclonal cell lines stably expressing Myc-tagged wild-type or mutant Chk2 were generated by calcium phosphate transfection (U-2-OS) or electroporation (HCT-15) of cells with empty pcDNA3-Myc vector or pcDNA3-Myc vector containing wild-type or mutant Chk2. A vector encoding puromycin resistance (pBabe-puro) was co-transfected, the cells were selected for neomycin (G418; 400 mg/ml) and puromycin (1 mg/ml) resistance and pooled when visible colonies emerged. Ionizing radiation was delivered by X-ray generator (RT100, Philips Medico; 100 kV, 8 mA, dose-rate 0.92 Gy/min) and cell extracts prepared 1 h later except where indicated otherwise.

Immunochemical techniques: Antibodies used in this study: Mouse monoclonal antibodies against human Cdc25A (DCS-122, DCS-124) and F-6 (Santa Cruz), Cdc25B (DCS-162, DCS-164) and Cdc25C (DCS-193) full length proteins fused to GST were generated by means of established hybridoma technology as described. Rabbit polyclonal antobody against Cdc25C (SC-327) were purchased from Santa Cruz. Antiserum against cdk2 Tyr15 was obtained from Calbiochem. Other mouse monoclonal antibodies: 12CA5 against the haemagglutinin epitope, DCS-60 and DCS-61 against human p21, 5D4 against human cyclin B1 and D2, HE12 and HE172 against hukan cyclin E, C23420 against human cyclin B1 (Transduction Laboratories). Rabbit polyclonal serum against human Chk1 was provided by S. Elledge. Protocols for immunoblotting, immunoprecipitation, immunostaining, and in vitro kinase assays were reported. Recombinant GST-Cdc25A used as a substrate for in vitro Chk1 kinase assay was purified as described. Dephosphorylation of cdk2 was initiated by addition of 10 mM EDTA and performed essentially as described [Blasina et al., Curr. Biol. 9:1(1999)].

Rabbit antisera to Cdk2 (M2), to Chk1 (FL-476), and mouse antibody to Cdc25A (F-6) were purchased from Santa Cruz. The latter antibody was used to detect endogenous Cdc25A protein. Rabbit antisera to Tyr15-phosphorylated Cdk1/Cdk2 was purchased from Calbiochem, and mouse monoclonal antibody to CD20 from Becton Dickinson. 9E10 antibody to Myc epitope was a gift from G. Evan. Mouse monoclonal antibodies to human Chk2 (DCS-270) and Cdc25A (DCS-127) were generated by standard hybridoma technology.

Flow cytometry: Conditions for single and multiple-parameter flow cytometry were described in detail. All analyses were performed on FACSCalibur flow cytometer (Becton Dickinson) using CellQuest and ModFit softwares.

Microinjection and in situ reporter assays: Asynchronous U-2-OS/C6C6 cells were either grown in the presence of tetracycline (2 μg/ml) or induced to express p53DD for 24 hours. Subsequently, the cells were microinjected with the mdm2/Luc reporter plasmid (25 μg/ml) and non-immune mouse IgG as a microinjection marker. After additional 18 hours, the cells were fixed and processed for combined anti-mouse IgG and anti-luciferase immunostaining as described.

Phosphatase assays: Cdc25 phosphatase activity was measured as an ability to activate cyclin B1/Cdc2. To obtain inactive cyclin B1/Cdc2 complexes, the U-2-OS cells were incubated in the presence of adriamycin (Calbiochem; 0.2 μg/ml) for 24 hours. Such treatment has been reported to induce inhibitory phosphorylation of Cdc2 on Thr14 and Tyr15, respectively (Poon et al., 1997). Inactive cyclin B1/Cdc2 complexes were immunoprecipitated with anti-cyclin B1 antibodies (200 μg of cell lysate per reaction). Cdc25 was immunoprecipitated in parallel using monospecific antibodies from 1 mg of cell extract treated as indicated. The beads from the cyclin B and Cdc25 immunoprecipitates were subsequently mixed in incubated in a final volume of 50 μl of a phosphatase buffer (20 mM Tris, pH 8.3; 150 mM NaCl; 2 mM EDTA; 0.1% Triton X-100; 5 mM DTT) at 30° C. for 1 hour the reaction was stopped by addition of kinase assay buffer and the activity of cyclin B/Cdc2 was assayed as described.

### Radioresistant DNA Synthesis Assay

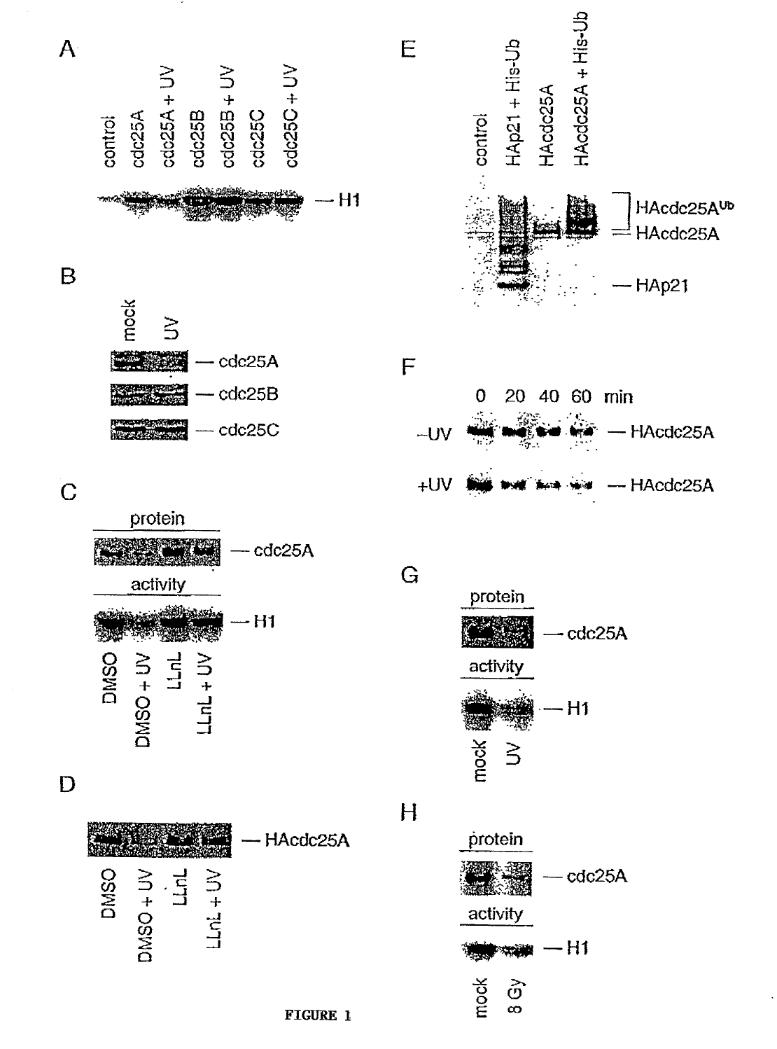

Inhibition of DNA synthesis after ionizing radiation was monitored as described in Lim et al, Nature 404, 613-617 (2000)) and Stewart et al (Cell 99, 577-587 (1999)). Cells were labelled for 24 h with 20 nCi/ml [¹⁴C]thymidine, followed by another 24 h-incubation in non-radioactive medium. Cells were then irradiated with either 0 Gy (control) or 10 Gy and 30 min post-irradiation, [³H]thymidine (2.5 mCi/ml) was added. After 15 min, cells were harvested in 0.25M NaOH, either directly or after isolation with anti-CD20-coupled Dynabeads as specified in Figure legends, and radioactivity measured in a liquid scintillation counter. The DNA synthesis was estimated by the ratios of [³H]/[¹⁴C] and expressed as a percentage of control values. Alternatively (FIG. 1a), the U-2-OS-derived B3C4 clone conditionally expressing HA-tagged Cdc25A was pre-labelled as above, but expression of the transgene was induced 1 h prior to irradiation with either 0, 5, 10 or 20 Gy immediately followed by addition of [³H]thymidine (2.5 mCi/ml). Cells were then labelled for 4 h and processed as above.

Alkaline elution. DNA single strand breaks were measured by alkaline elution using H202-treated L1210 cells as internal standard exactly as described [Sehested et al., Cancer Res. 58, 1460 (1998)]. Relative DNA single strand break yields were calculated using the SAS statistical software package.

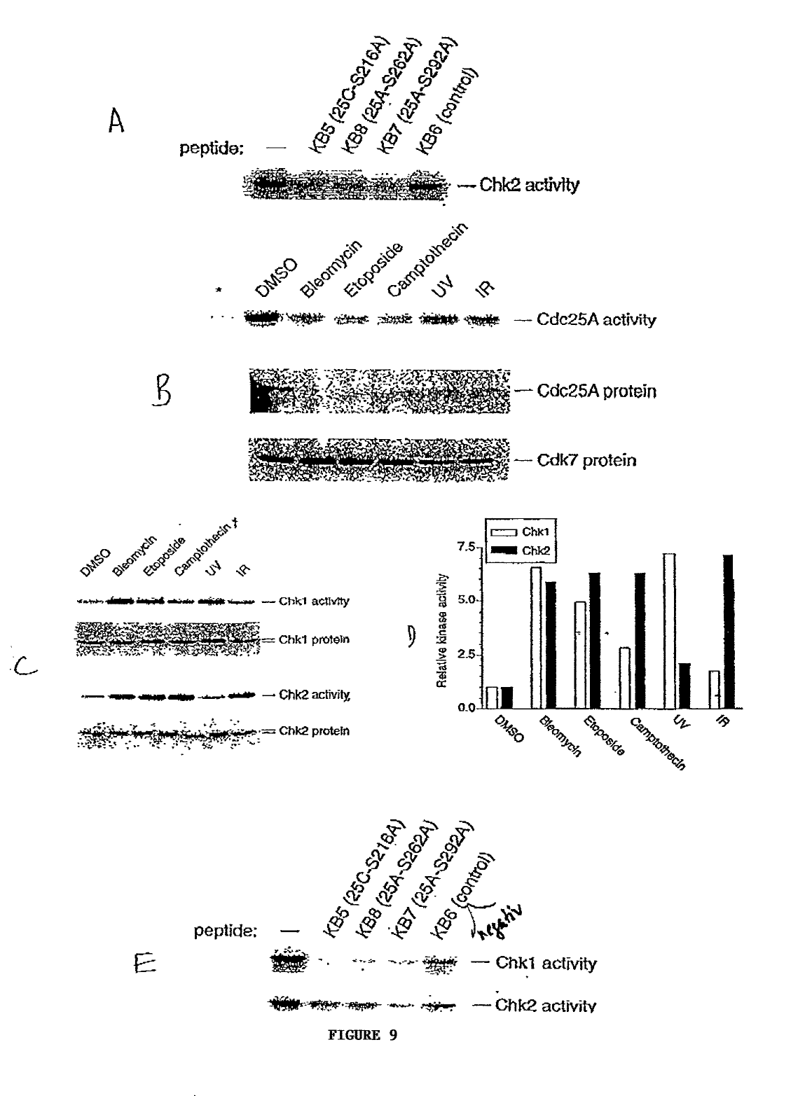

In vitro kinase assay. 500 μg of whole cell lysate (WCE) was immunoprecipitated with protein G-sepharose beads coupled to Chk2 antibody (DCS-270: 2 μl ascites per reaction) by incubation for 2 hours at 4° C. Beads were washed 3 times in IP buffer and two times in kinase assay buffer (KAB). The kinase reaction was performed for 1 hour at 30° C. in 30 μl kinase reaction mixture (KRM) using myelin basic protein (MBP; Sigma #M2016) as a substrate and containing 10 μM of KB peptide. The reaction was terminated by addition of 15 μl of hot SDS sample buffer. Sample (25 μl) were resolved by 15% SDS-PAGE, the gel dried and exposed to a phosphoroimager screen. The cells were exposed to chemotherapeutic agents, ionizing radiation and UV light. Peptides based on the Ser262 and Ser292 motifs of Cdc25A were used with a control peptide based on Ser216 of Cdc25C. Results are set out in FIG. 9.

IP buffer—50 mM HEPES; pH7.5; 150 mM NaCl; 1 mM EDTA; 2.5 mM EGTA; 10% glycerol; 0.1% Tween 20; 2 μg/ml leupeptin; 2 μg/ml aprotinin; 0.1mM phenylmethylsulfonyl fluoride (PMSF); 10 mM β-glycerophosphate; 1 mM NaF; 0.1 mM Na₃VO₄.

KAB—50 mM HEPES; pH7.5; 10 mM MgCl₂; 2.5 mM EGTA; 10 mM β-glycerophsohate; 1 mM DTT; 0.1 mM Na₃VO₄.

KRM—15 μl KAB; 9 μl cold ATP; 50 μl MBP; 111 ³²P-gamma ATP.

The results from these assays indicate that these peptides are able to inhibit Chk1 and Chk2 kinases under in vitro conditions. This inhibition is most likely due to direct competition with the substrate (in this case myelin basic protein, MBP), thus this is the so-called mode of action of the peptides. Importantly, these assays demonstrated inhibition by the peptides in vitro, a prerequisite for an in vivo effect (i.e. abrogation of Cdc25A degradation). Serine 216 is found in Cdc25C, and the peptide containing this residue (both wild type and the alanine mutant) was included as a control (see Suganuma et al., Cancer Res. 59(23):5887-91, 1999).

### Results

It has been firmly established that the cell cycle arrest at the G2 checkpoint requires inactivation of cdk1 by its phosphorylation on Tyr15, which is achieved by functional sequestration of the Cdc25C phosphatase. We therefore began our search for rapid G1/S checkpoint responses, which might precede the activation of the p53/p2l pathway by analysis of the functional status of the whole family of Cdc25 phosphatases.

To study the dynamic changes in Cdc25 phosphatase activities in response to DNA damage, we exposed exponentially growing human U-2-OS cells (wild-type for p53) to a single non-lethal dose of ultraviolet light (15 J/m²), a model mutagen commonly used to study checkpoint pathways including the p53/p21 axis. While the specific activities associated with Cdc25B and Cdc25C remained as high as in non-irradiated cells, the Cdc25A phosphatase activity rapidly declined and stayed low for at least two hours after UV irradiation (FIG. 1A). This was accompanied by quantitative elimination of the Cdc25A protein following UV irradiation, again in sharp contrast to Cdc25B and Cdc25C proteins, which under identical experimental conditions remained unchanged (FIG. 1B). Thus, in terms of protein abundance and activity, Cdc25A appeared to represent a selective target within the Cdc25 family in response to UV. Since steady-state levels of Cdc25A mRNA were not affected by UV (negative data not shown), we reasoned that the UV-induced downregulation of Cdc25A in our experiments might reflect changes of its protein turnover. Indeed, addition of the proteasome inhibitor LLnL immediately after UV irradiation entirely prevented the reduction of protein level as well as phosphatase activity of both the endogenous (FIG. 1C), and the ectopic Cdc25A expressed from a heterologous promoter (FIG. 1D). Moreover, using a standard in vivo ubiquitination assay, we could detect abundant high molecular weight species resulting from polyubiquitinated Cdc25A protein (FIG. 1E, lane 4). Finally, pulse-chase experiments directly demonstrated shortening of the Cdc25A protein half-life in U-2-OS cells exposed to UV when compared to non-irradiated cells (FIG. 1F). In summary, these experiments showed that Cdc25A could be targeted by the ubiquitin/proteasome pathway and that its protein turnover could be conditionally accelerated as a response to UV-induced DNA damage.

To explore the possibility that the Cdc25A protein stability represents a previously unrecognized target of a checkpoint response to genotoxic stress, we first tested whether the data obtained in an established tumour cell line can be recapitulated also in normal diploid cells. We exposed the human fibroblast strain, IMR90, to the same dose of UV and measured the impact on Cdc25A protein levels and activity. Similarly to U-2-OS cells, irradiated fibroblasts sharply downregulated the Cdc25A protein and phosphatase activity (FIG. 1G). Furthermore, when we exposed the IMR90 cells to a different source of DNA damage such as ionizing radiation, we also observed rapid disappearance of the Cdc25A protein and sharp decline of its associated phosphatase activity. We concluded that in human cells, DNA damage triggers a specific checkpoint response, which induces rapid destruction of Cdc25A.

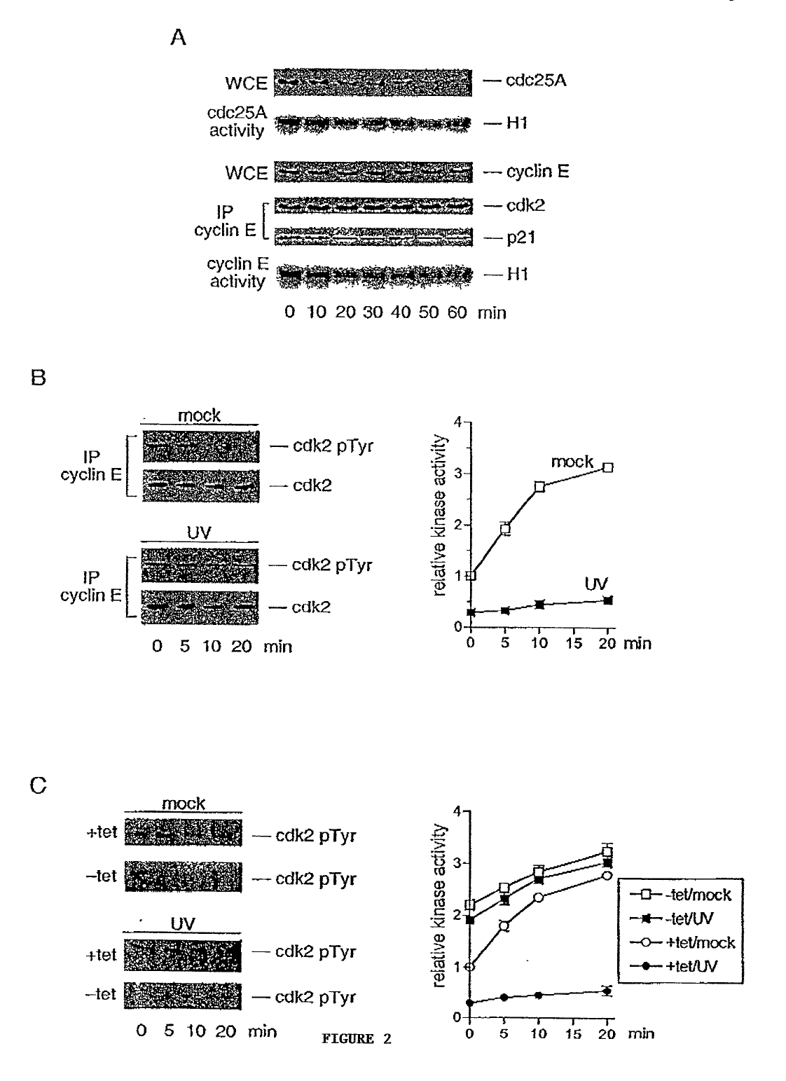

To identify the downstream target(s) of the observed inactivation of the Cdc25A phosphatase, we next determined the detailed kinetics of the Cdc25A protein and activity downregulation after DNA damage. Both Cdc25A protein and its associated phosphatase activity began to decline at 20 minutes and reached the nadir around 50 minutes after exposure to UV light (FIG. 2A). As a next step, we measured within the same time-frame the specific activities of cyclin/CDK complexes known to regulate the G1/S transition and suggested previously to be activated by Cdc25 phosphatases under various experimental conditions. Protein levels of the D-type cyclins and their associated kinase activities towards the retinoblastoma protein remained unchanged. On the contrary, whereas cyclin E protein levels remained also constant, its associated histone H1 kinase activity was rapidly inhibited with dynamics closely following the UV effect on the Cdc25A phosphatase (FIG. 2A, bottom panel). This inhibition could not be explained by p21 recruitment into the cyclin E/cdk2 complexes, since co-immunoprecipitation experiments revealed no increase in association of cyclin E with p21 (FIG. 2A). Together with the fact that also the ability of cyclin E to interact with its catalytic partner cdk2 was not changed (FIG. 2A), and that the overall phospho-tyrosine amount associated with cdk2 complexes increased after UV irradiation, these data strongly indicated that the rapid inhibition of cyclin E/cdk2 in response to UV light could reflect insufficient removal of inhibitory phosphates from Tyr15 located within the ATP-binding lobe of cdk2.

To test whether the inhibition of cdk2 activity was causally linked to the UV-induced destruction of Cdc25A, we employed a modified in vitro assay allowing us to directly quantify the cyclinE/cdk2 activity as a function of the extent of its inhibitory tyrosine phosphorylation. This approach revealed that lysates prepared from the mock-treated cells supported progressive tyrosine dephosphorylation (FIG. 2B, upper panels) and corresponding superactivation (FIG. 2B, right) of the cyclin E/cdk2 complexes.

On the contrary, lysates from the cells exposed to UV light significantly lost their ability to dephosphorylate cyclin E/cdk2 (FIG. 2B, lower panels), the activity of which remained very low throughout the assay period (FIG. 2B, right). Two subsequent experiments confirmed that the differences between the mock- and UV-treated cells were indeed due to the UV-induced inhibition of the phosphatase activity associated with Cdc25A: firstly, we could demonstrate that the addition of sodium vanadate, a potent inhibitor of multiple tyrosine phosphatases including Cdc25 into the reaction completely prevented the ability of mock-treated cells to dephosphorylate and superactivate cyclin E/cdk2 (FIG. 2C). Secondly, using our conditionally manipulatable cell lines, we were able to induce the expression of the ectopic Cdc25A to sufficiently high levels that saturated the cellular capacity to efficiently degrade it. Such interference with the UV-induced Cdc25A degradation lead to substantial and immediate increase of cyclin E/cdk2-associated kinase activity isolated from the non-irradiated cells (FIG. 2D), in contrast to the gradual accumulation of cdk2 activity dependent on its dephosphorylation by the endogenous phosphatase (FIGS. 2B and 2D). Importantly, the presence of elevated ectopic Cdc25A could also quantitatively restore the ability of the UV-treated cells to dephosphorylate cdk2 and dramatically increased the cyclin E/cdk2 kinase activity compared to the UV-treated cells without Cdc25A pre-induction (FIG. 2D). A similar, albeit somewhat less pronounced effect was also observed when cyclin A-associated kinase activity was assayed under the same experimental conditions (data not shown). Taken together, these data demonstrate that the reduced level and activity of the Cdc25A phosphatase in UV-irradiated cells directly contribute to the increase and maintenance of the tyrosine phosphorylation and the subsequent inhibition of cdk2-containing complexes, whose enzymatic activities are critically important for supporting DNA replication.

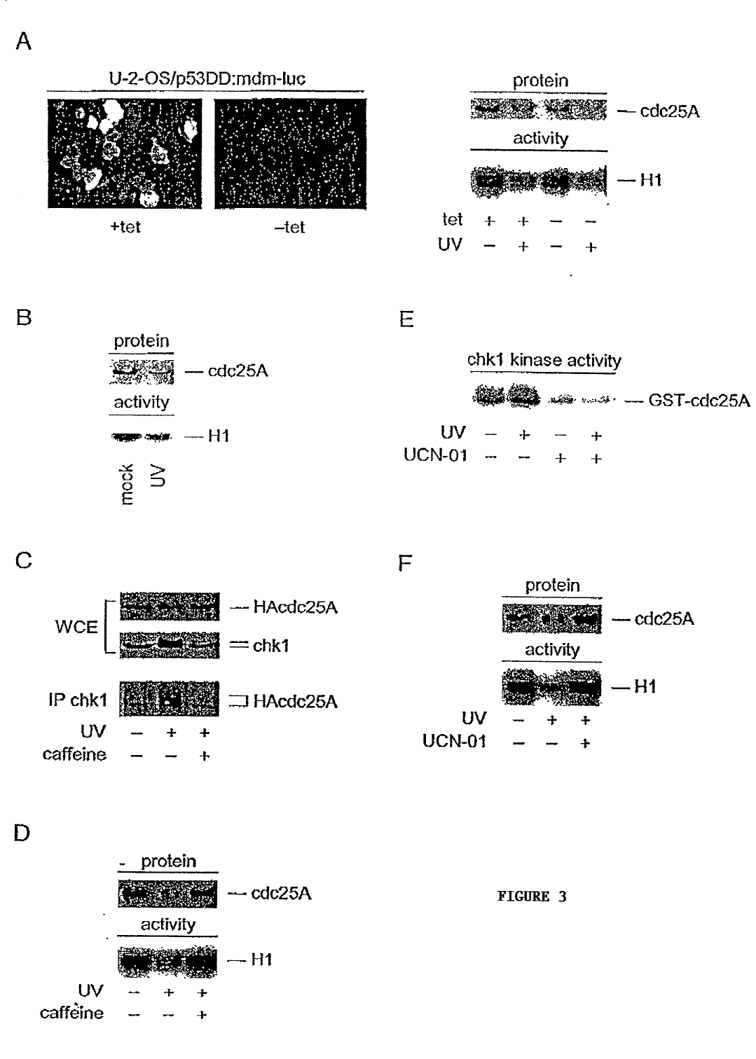

Studies of mammalian cells in response to genotoxic stress including UV-induced DNA damage have implicated the p53 tumour suppressor as a key factor controlling the cell cycle arrest in both G1 and G2 phases. Moreover, p53 was proposed to directly participate on functional sequestration of the mitotic Cdc25C by regulating expression of the 14-3-3s protein. To examine whether p53 is essential also for the UV-induced degradation of Cdc25A observed in our experiments, we established a U-2-OS subline conditionally expressing a dominant-negative allele of p53 (p53DD). Consistent with previously reported effects of this p53 mutant, we could demonstrate that induction of p53DD in our cell line quantitatively abolished the p53-dependent transactivation potential (FIG. 3A, left panels) and expression of endogenous p53 targets such as p21. Importantly, following UV irradiation, both p53 wild-type and p53DD expressing cells downregulated Cdc25A protein and phosphatase activity to very similar extent (FIG. 3A, right panels). Consistently, in UV-exposed SAOS-2 human osteosarcoma cell line, which completely lost the expression of the endogenous p53, the Cdc25A protein was degraded and its activity downregulated with the kinetics indistinguishable from cells with wild-type and functional p53 (FIG. 3B). Thus, the checkpoint response activated by UV irradiation evokes rapid destruction of the Cdc25A protein independently on p53-mediated transcriptional activity.

One previously established mode of regulating the mitotic Cdc25 phosphatase in response to DNA damage is its phosphorylation the Chk1 protein kinase. Although this G2 checkpoint does not operate via enhanced degradation of the targeted phosphatase, the Chk1 kinase was an attractive candidate for mediating also the early effects at G1/S in our model, since Chk1 can phosphorylate also the other members of the Cdc25 family, at least in vitro. In support of a possible involvement of the Chk1 kinase, we observed that exposure to UV light induced a pronounced shift of the Chk1 protein on the SDS gel (FIG. 3C, middle panel), indicating its activatory phosphorylation. This was accompanied by a dramatic increase in Chk1/Cdc25A association (despite the reduction of the overall level of the Cdc25A protein), an effect which could be quantitatively prevented by short pre-treatment of the cells with caffeine (FIG. 3C, lower panel), a compound which can interfere with activation of the Chk1 kinase presumably through the inhibition of the upstream operating ATM and/or ATR kinases. Consequently, exposure of cells to caffeine prior to UV irradiation prevented the shift of the Chk1 protein (FIG. 3C, middle panel, lane 3), abolished the protein destruction and loss of phosphatase activity of Cdc25A (FIG. 3D), and restored the half-life of the Cdc25A protein back to the values measured in non-irradiated cells. We noticed that the Chk1-associated Cdc25A protein migrated more slowly on SDS gels (FIG. 3C, middle panel, lane 2), suggesting that the Chk1 kinase may phosphorylate Cdc25A and thereby prime it for rapid destruction.

To exclude any adverse, Chk1-independent effect of caffein, and to directly prove that the induction of the Chk1 kinase activity represents an essential step in mediating the degradation of the Cdc25A phosphatase after DNA damage, we employed a newly discovered and highly specific Chk1 inhibitor, UCN-01. In a reconstituted in vitro Chk1-kinase assay using purified GST-tagged Cdc25A as a substrate we observed that the relatively low but measurable Chk1 kinase activity was indeed significantly elevated in cells exposed to UV light (FIG. 3E, lanes 1 and 2). Importantly, both basal and the UV-induced Chk1 activities were completely inhibited by adding UCN-01 into the kinase reaction (FIG. 3E, lanes 3 and 4). Moreover, treatment of cells with UCN-01 at the time of UV irradiation completely abolished the degradation of the endogenous Cdc25A protein and allowed maintenance of high Cdc25A-associated phosphatase activity (FIG. 3F). In summary, these data provide the first direct in vivo evidence for a functional link between the Chk1 kinase operating upstream of the Cdc25A degradation after UV-induced DNA damage.

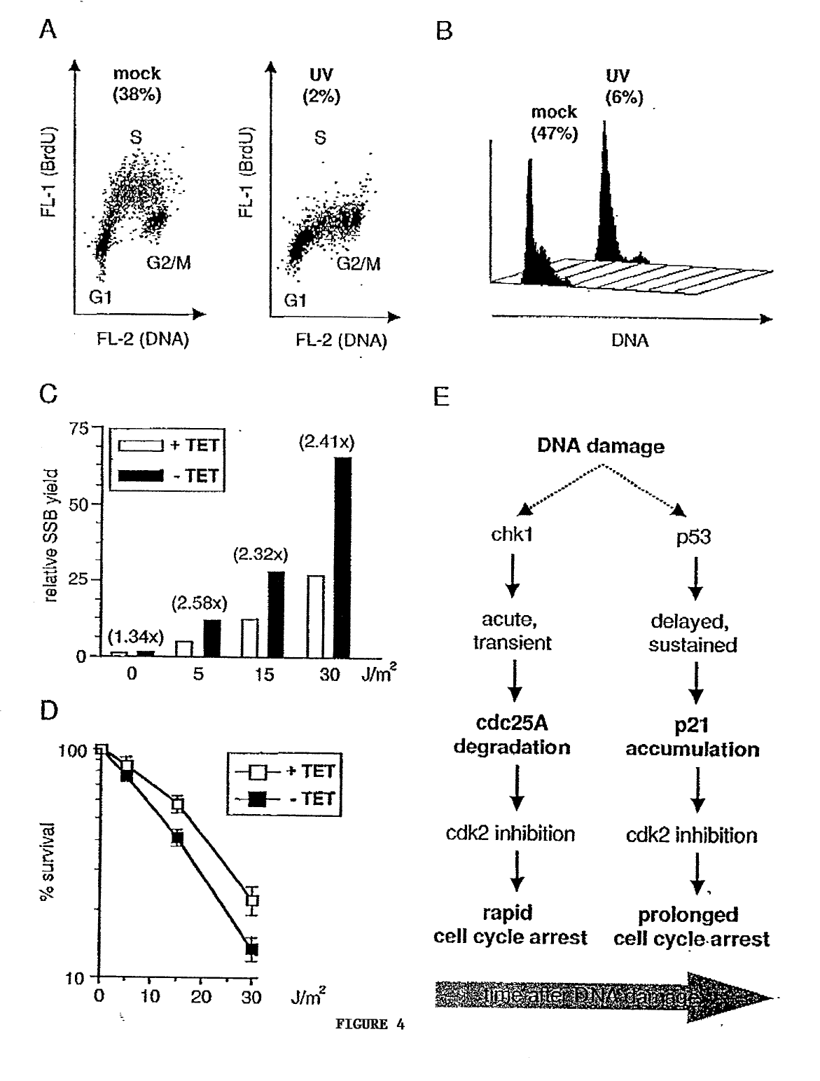

In order to link the observed Chk1-dependent downregulation of Cdc25A to the cell cycle arrest during the DNA damage-induced G1 checkpoint, we studied the cellular response of our model U-2-OS cells to UV light. Short pulses with BrdU revealed that the ongoing DNA synthesis in exponentially growing cells was dramatically inhibited within 1-2 hours following UV irradiation (FIG. 4A). Subsequent analyses of the kinetics of the G1/S progression in cells released from the nocodazole arrest showed that also the S-phase entry was effectively blocked after UV irradiation (FIG. 4B). These findings were consistent with the known requirement of cdk2 activity for both S-phase entry and progression. As a next step we followed the irradiated cells in the time course and found that between 16 and 24 hours after exposure to UV light, the cells progressively resumed DNA replication and progression through the cell cycle. These data indicate that the doses of the UV light used in our experiments generated reversible cell cycle arrest.

The predicted impact of bypassing the UV-induced degradation of Cdc25A was then assessed by the alkaline elution assay measuring DNA single-strand breaks as a consequence of continued, uninhibited activity of the DNA replication machinery in the UV-damaged cells. Within a broad range of UV doses, the regulatable ectopic expression of Cdc25A significantly enhanced the formulation of the DNA single-strand breaks (FIG. 4C), likely resulting from collisions of the ongoing extension of the replication forks with the UV-induced DNA crosslinks. The ectopic Cdc25A in the absence of UV irradiation did not cause any DNA damage measurable in this assay (FIG. 4C), indicating that the abundance of the Cdc25A phosphatase represents a rate limiting factor determining the extent of DNA synthesis upon UV-induced DNA damage. Thus, the rapid elimination of Cdc25A evokes a cell cycle arrest permissive for repair of the DNA crosslinks commonly caused by UV, and at the same time protecting the cells from formulation of the DNA strand breaks. Finally, consistent with the third prediction for a checkpoint response, a series of colony formation assays revealed that a short induction of ectopic Cdc25A precluding efficient downregulation of the cellular Cdc25A activity by UV (see also FIG. 2B, D and FIG. 4C) significantly reduced the survival of the irradiated cells (FIG. 4D).

Taken together, these results uncover a novel mechanism of cellular defense to genotoxic stress. The salient features of this G1/S checkpoint response are:

(1) Proteasome-dependent destruction of the Cdc25A phosphatase as a mediator affecting the extent of tyrosine phosphorylation and activity of cdk2.

(2) Independence on the p53/p21 checkpoint pathway.

(3) Requirement for the integrity of the Chk1 kinase operating upstream of the Cdc25A degradation.

(4) A rapid execution of the response.

Based on the characteristics of the rapid response through degradation of Cdc25A reported here, and the established response to UV irradiation via activation of p53, we propose a working model of a ‘two-wave’ checkpoint response to UV-induced DNA damage (FIG. 4D). By virtue of its rapid kinetics, the Cdc25A-mediated mechanism can significantly contribute to genomic stability, by timely imposing a cell cycle block and preventing excessive damage of the genome before the activated p53/p21 pathway ensures a more sustained proliferation arrest. Deregulation of either mechanism can cause genomic instability, as documented for p53 mutations, and plausible when Cdc25A, a bona-fide protoncogene in transformation assay, becomes overexpressed. Overabundance of Cdc25A found in subsets of aggressive human cancers possibly prevents its timely degradation in response to DNA damage, and provides a growth advantage by escape from G1/S arrest, and propagation of genetic abnormalities.

In further investigations, we examined whether degradation of Cdc25A affects the IR-induced S-phase checkpoint, we measured DNA synthesis in human U-2-OS/B3C4 cells engineered to conditionally express ectopic Cdc25A in a tetracycline-repressible manner. Whereas endogenous Cdc25A was rapidly degraded in response to a range of IR doses, accompanied by increasing dose-dependent inhibition of DNA synthesis (FIG. 5a), transient elevation of the ectopic HA-Cdc25A protein to levels sufficiently high to saturate the cellular capacity to efficiently degrade it resulted in abrogation of the S-phase checkpoint (FIG. 5a). The degree of RDS in U-2-OS/B3C4 cells unable to downregulate Cdc25A protein was comparable to that seen in cells from A-T patients.

If destruction of Cdc25A was a genuine part of the S-phase checkpoint, two predictions should be fulfilled. First, it has been well established that the S-phase inhibition of DNA replication is a transient response lasting only several hours following exposure to IR. Our kinetic measurements showed that downregulation of Cdc25A was nearly completed 30 minutes after irradiation but recovered to the levels seen in non-irradiated cells during the following 4-8 hours (FIG. 5b). Under the same experimental conditions, the protein level and/or activity of Cdc25B and Cdc25C showed only little variation. The period of the maximal Cdc25A downregulation between 1-3 hours after exposure to IR correlated with increased inhibitory phosphorylation of Cdk2 on tyrosine 15, reduction of the S-phase-promoting cyclin E/Cdk2 kinase activity, and nearly 50% inhibition of DNA synthesis (FIG. 5b). Second, if the major cell cycle-inhibitory consequence of the IR-induced Cdc25A downregulation was Cdk2 inhibition, interference with the Cdk2 inhibitory phosphorylation should mimic the effect of Cdc25A overexpression and abrogate the S-phase checkpoint. Indeed, brief conditional expression of the Cdk2AF allele where the inhibitory threonine 14 and tyrosine 15 were substituted by alanine and phenylalanine, respectively, resulted in RDS, the extent of which was comparable to that achieved by transient overexpression of Cdc25A (FIG. 5c). Simultaneous expression of both Cdc25A and Cdk2AF did not further increase the degree of RDS (FIG. 5c) indicating that both proteins function within the same pathway.

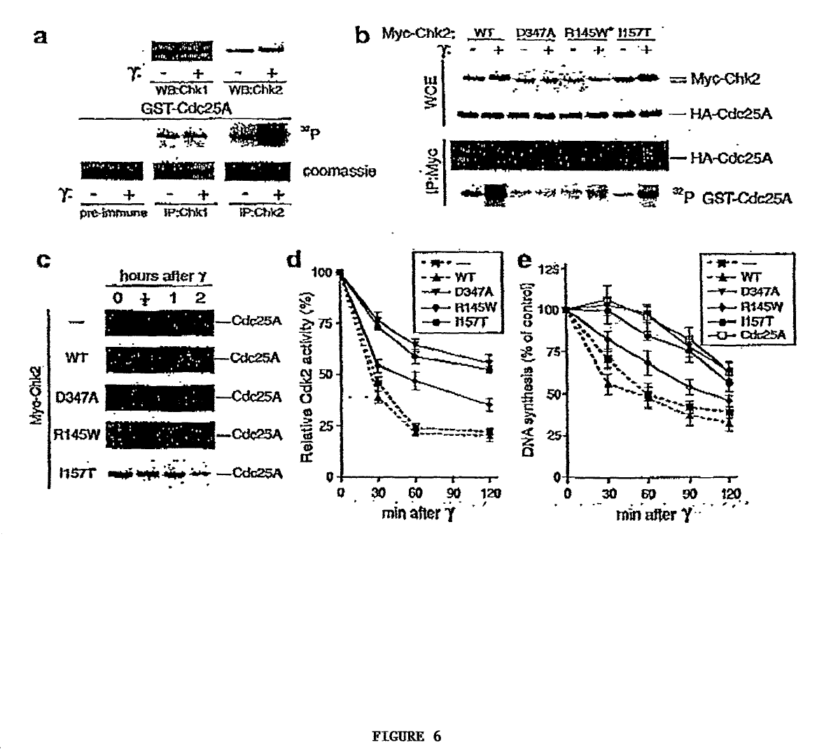

Ultraviolet light (UV)-induced downregulation of Cdc25A requires rapid activation of Chk1, a kinase which together with Chk2 represent a class of key signal transducers implicated in checkpoint pathways activated by damaged or unreplicated DNA. To assess whether Chk1 or Chk2 may regulate abundance of Cdc25A upon IR, we measured their abilities to phosphorylate Cdc25A. Whereas the activity of cellular Chk1 remained low until several hours after irradiation (FIG. 6a), Chk2 became activated rapidly, as manifest by its slower electrophoretic mobility and enhanced phosphorylation of GST-Cdc25A one hour after irradiation (FIG. 6a). These results suggested that Chk2 but not Chk1 becomes activated with kinetics expected for a link between IR-induced DNA damage and the rapid S-phase inhibition. Detailed time-course measurements revealed that the kinetics of IR-induced Chk2 activity closely paralleled the Cdc25A protein and phosphatase activity loss, downregulation of cyclin E/Cdk2 by inhibitory phosphorylation, and decrease of DNA synthesis (FIG. 5b).

To gain mechanistic insight into the emerging link between Chk2 and Cdc25A, we assessed responsiveness to IR of a panel of Myc-tagged forms of human Chk2 transiently transfected into U-2-OS/B3C4 cells, including the engineered catalytically-inactive D347A mutant, and R145W and I157T alleles mutated in the putative protein-interaction FHA domain. The latter two mutants were identified in sporadic colon cancer and as a germ-line mutation in the cancer-prone Li-Fraumeni syndrome, respectively, raising the possibility that Chk2 may represent a novel tumour suppressor. Upon exposure of transfected cells to IR, Myc-tagged wild-type Chk2 became rapidly shifted into a slower-migrating form, accompanied by an increase in its ability to physically interact with transiently elevated HA-Cdc25A in vivo, and phosphorylate GST-Cdc25A in vitro (FIG. 6b). In contrast, basal activities of the three mutants of Chk2 were barely detectable in non-irradiated cells and remained low even after exposure to IR (FIG. 6b). Interestingly, the catalytically inactive D347A mutant retained the ability to bind HA-Cdc25A after IR (FIG. 6b), implying that the interaction of Chk2 with its downstream substrate(s) is mediated via its modification by some upstream component of the checkpoint pathway, independent of Chk2 autocatalytic activity. On the contrary, the FHA domain-mutated, cancer-associated mutants of Chk2, R145W and I157T, were unable to bind HA-Cdc25A (FIG. 6b). The I157T mutant preserved the IR-induced mobility shift (FIG. 6b) suggesting preserved autocatalytic activity and/or intact modification by upstream regulators, while the shift of R145W was not detected, possibly due to the lower protein stability of this mutant. Hence, the major defect of the tumour-associated Chk2 proteins appears to reside in their lost ability to bind and phosphorylate the downstream components of the IR-induced checkpoint pathways such as Cdc25A.

A plausible interpretation of the above results could be that Chk2 and Cdc25A operate on a common linear pathway directly involved in establishing the S-phase checkpoint after IR-induced DNA damage. To validate this hypothesis, we transfected U-2-OS cells with plasmids encoding the Chk2 alleles described in FIG. 6b together with a gene coding for puromycin resistance. After one week of puromycin selection, the resulting polyclonal cell populations were 60-80% homogeneous in expressing the individual Chk2 transgenes. The ectopically expressed proteins exceeded 3-5 fold the levels of endogenous Chk2, localized to cell nuclei, and did not influence cell cycle progression in the absence of DNA damage. However, while cells transfected with wild-type Chk2 or empty plasmid efficiently degraded Cdc25A when exposed to IR (FIG. 6c), cells expressing any of the Chk2 mutants retained substantial amounts of Cdc25A when irradiated (FIG. 6c). These differential abilities to modulate the steady-state levels of Cdc25A protein had a direct impact on the S-phase-promoting Cdk2 activity, which sharply declined in control or wild-type Chk2-transfected cells but remained elevated in cells expressing the Chk2 mutants (FIG. 6d). Consequently, whereas the cells transfected with empty vector or wild-type Chk2 responded by inhibiting DNA synthesis, cells expressing the Chk2 mutants, unable to degrade Cdc25A and inhibit Cdk2 activity, failed to impose the S-phase blockade and exhibited RDS comparable to cells conditionally overexpressing Cdc25A (FIG. 6e). Thus, genetic lesions interfering with the catalytic activity of Chk2 or its interaction with Cdc25A resulted in Chk2 variants functioning in a dominant-negative manner. Expression of such mutants abrogated the S-phase checkpoint, supporting the notion that Chk2 operates upstream of Cdc25A and Cdk2 within a common pathway.

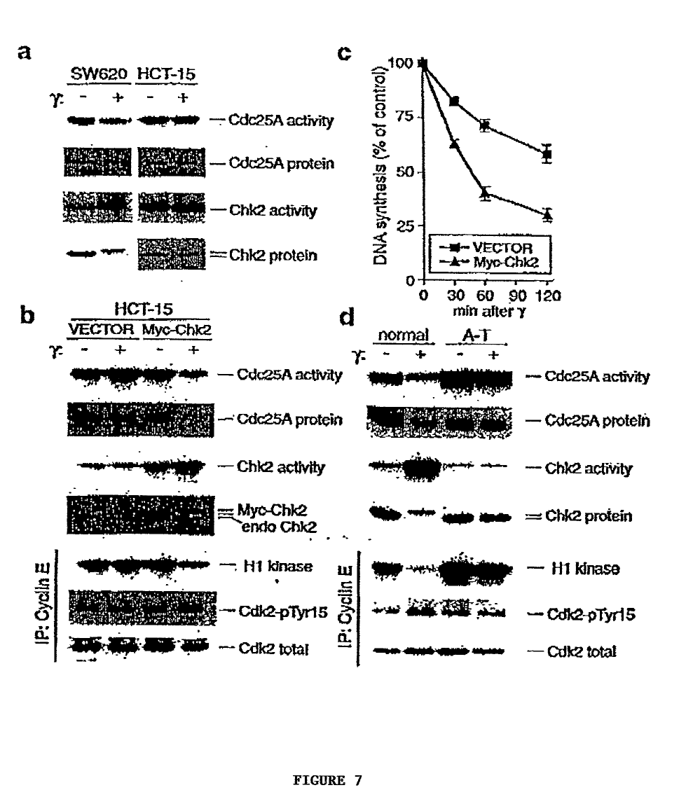

In addition to Cdc25A, Chk2 also efficiently phosphorylates the p53 tumour suppressor in response to DNA damage. However, it is the former substrate which appears relevant for the S-phase arrest induced by IR, since p53 controls the G1 and sustained G2/M, rather than S-phase checkpoint, RDS occurs independently of p53 status, and p53 stabilization and accumulation of its downstream targets such as the p21 Cdk inhibitor significantly lagged behind the Cdc25A degradation and S-phase inhibition in response to IR. Consistent with such interpretation, rapid degradation of Cdc25A and loss of its phosphatase activity followed exposure to IR in SW620 (wild-type for Chk2) but not in the HCT-15 cells expressing only mutant Chk2 (FIG. 7a). The lack of wild-type Chk2 expression in HCT-15 cells was confirmed by cDNA sequencing. Notably, both of these colon cancer cell lines harbour mutant p53. Reintroduction of the wild-type Chk2 into HCT-15 cells to the level exceeding that of the endogenous mutated protein restored the S-phase checkpoint response including destruction of Cdc25A protein and downregulation of its phosphatase activity (FIG. 7b), increased inhibitory phosphorylation of Cdk2 and downregulation of cyclin E/Cdk2 kinase activity (FIG. 7b), and the ability to arrest S-phase progression after exposure to IR (FIG. 7c). These results further support Chk2 as a mediator of rapid destruction of Cdc25A in response to IR, in a p53-independent S-phase checkpoint protecting cells against RDS.

The phenomenon of RDS has been most thoroughly characterized in cells with defective function of the ATM gene. Stimulated by our observation that deregulation of Chk2 and/or Cdc25A also led to RDS, and by the recent reports that ATM directly activates Chk2, we compared the response of the Chk2-Cdc25A-Cdk2 pathway in lymphoblasts derived from A-T patients to those isolated from normal individuals. Unlike normal lymphoblasts, irradiated A-T cells were unable to activate Chk2 and downregulate Cdc25A protein and activity (FIG. 7d). Consequently, exposure of A-T lymphoblasts to IR caused neither increase in Cdk2 tyrosine 15 phosphorylation, nor inhibition of cyclin E/Cdk2 kinase activity (FIG. 7d), consistent with the well documented RDS phenotype in these cells.

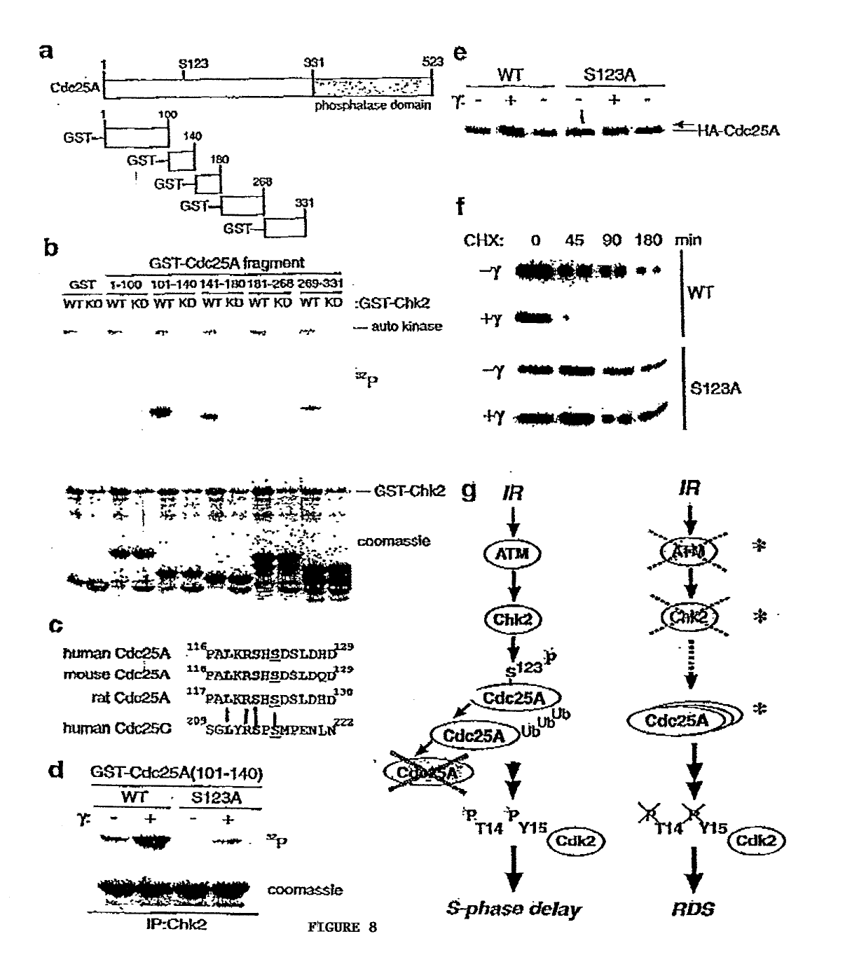

Signals from damaged DNA are often propagated through phosphorylation cascades, and the major residues required for IR-induced and ATM-mediated phosphorylation of Chk2 have recently been identified. To gain mechanistic insight into the functional interplay between Chk2 and Cdc25A in the S-phase checkpoint, we sought to identify the Cdc25A residue(s) directly phosphorylated by Chk2, presumably priming the phosphatase for rapid destruction following IR-induced DNA damage. A series of glutathione-S-transferase (GST)-coupled fragments derived from the Cdc25A regulatory domain (FIG. 8a) were constructed and subjected to phosphorylation by purified GST-Chk2 in an vitro kinase assay. The fragment spanning amino acids 101-140 was strongly phosphorylated by wild-type but not catalytically inactive Chk2 (FIG. 8b), and sequence flanking serine 123 of human Cdc25A, conserved in diverse mammalian species, matched the criteria proposed for a Chk2/Chk1 consensus site identified in other proteins such as Cdc25C (FIG. 8c). Substitution of the serine 123 to alanine almost completely abolished the capability of the IR-activated Chk2 to phosphorylate the corresponding GST-Cdc25A fragment (FIG. 8d). Mutation of serine 123 in the context of the full-length Cdc25A resulted in a protein which, unlike wild-type Cdc25A, did not undergo an IR-induced shift when separated on an SDS gel (FIG. 8e), strongly suggesting that also in vivo the S123A substitution abolished an important IR-dependent modification of Cdc25A. Significantly, S123A mutation rendered the Cdc25A protein resistant to IR-induced degradation under conditions when a comparable amount of moderately overexpressed wild-type Cdc25A was still effectively degraded in vivo (FIG. 8f). We concluded that Chk2-dependent phosphorylation of Cdc25A on serine 123 represents a critical step in promoting its rapid destruction in response to IR-induced DNA damage.

Based on these invetsigations, we propose that the ATM-Chk2-Cdc25A-Cdk2 pathway described here may provide the long-sought molecular explanation of the defense mechanism protecting human cells against RDS. The key components and events along this pathway in cells proficient in the S-phase checkpoint are schematically outlined in FIG. 8g (left). The biological and pathophysiological relevance of this mechanism is further supported by the fact that tumour-associated defects of any of its major components cause RDS (FIG. 8g, right), and may predispose to, or promote tumourigenesis. Cells undergoing DNA replication are particularly vulnerable to genotoxic stress including IR, and the rapid execution of the checkpoint pathway(s) targeting Cdc25A for degradation and resulting in an instant inhibition of the replication-promoting Cdk2 activity may represent the initial defense barrier, which inhibits DNA synthesis to allow for efficient repair. Cdc25A extends the list of important checkpoint mediators targeted by Chk2, currently encompassing the mitotic activator Cdc25C, and the p53 and BRCA1 tumour suppressors, thereby implicating Chk2 in a complex network controlling G1, S, and G2/M checkpoints, as well as DNA repair. Such a central role in protecting genome integrity is also consistent with the recently proposed candidacy of Chk2 for a novel tumour suppressor whose mutations may predispose to tumourigenesis in diverse tissues, as seen in Li-Fraumeni families. Our finding that the tumour-associated Chk2 alleles are indeed loss-of-function mutants provides the missing functional evidence strongly indicating that Chk2 is a bona-fide tumour suppressor.

### References

The references mentioned herein including the following specific research articles are all expressly incorporated by reference.

Matsuoka et al, Science, 282:1893, 1998.

Chaturvedi et al, Oncogene, 18:4047, 1999.

Blasina et al, Curr. Biol., 14:1, 1999.

The invention has been described in detail with reference to preferred embodiments thereof. However, it will be appreciated that those skilled in the art, upon consideration of the disclosure, may make modification and improvements within the spirit and scope of the invention.

What is claimed is:

1. Use of a substance which is capable of inhibiting the interaction of Cdc25A and Chk1 or Chk2 for the preparation of a medicament for the treatment of cancer or a hyperproliferative disorder.

2. The use of claim 1, wherein the substance inhibits the binding of Cdc25A to Chk1 or Chk2 or the phosphorylation of Cdc25A by Chk1 or Chk2.

3. The use of claim 1 or claim 2, wherein the hyperproliferative disorder is psoriasis, arteriogenesis or inflammation.

4. The use of any one of claims 1 to 3, wherein the substance is administered in combination with chemotherapy or radiotherapy.

5. The use of claim 4, wherein the chemotherapy includes the administration of a DNA topoisomerase toxin.

6. The use of claim 5, wherein the DNA topoisomerase toxin is an anthracycline, an epipodophyllotoxine or a camptothecin derivative.

7. The use of claim 4, wherein the radiotherapy includes the use of γ-radiation.

8. The use of any one of the preceding claims, wherein the substance comprises:
(a) a peptide fragment of between 5 and 30 amino acids which has at least 80% sequence identity with a corresponding sequence of Cdc25A, the fragment including a serine residue at a position corresponding to amino acid Ser123 or Ser262 or Ser292 or Ser504 in Cdc25A; or,
(b) a derivative of peptide fragment (a); or
(c) a substance which is peptide fragment (a) or derivative (b) linked to a coupling partner.

9. The use of claim 8, wherein the substance is linked to coupling partner.

10. A substance having the property of binding to Chk1 or Chk2 and inhibiting the phosphorylation of Cdc25A by the Chk1 or Chk2, the substance comprising:
(a) a peptide fragment of between 5 and 30 amino acids which has at least 80% sequence identity with a corresponding sequence of Cdc25A, the fragment including a serine residue at a position corresponding to amino acid Ser123 or Ser262 or Ser292 or Ser504 in Cdc25A; or,
(b) a derivative of peptide fragment (a); or
(c) a substance which is peptide fragment (a) or derivative (b) linked to a coupling partner.

11. An isolated nucleic acid molecule encoding the substance of claim 10.

12. An expression vector comprising the nucleic acid of claim 11, operably linked to sequences to direct its expression.

13. A host cell transformed with the expression vector of claim 12.

14. A method of producing the substance of claim 10, the method comprising culturing the host cells of claim 13 and isolating the substance thus produced.

15. The substance of claim 10 for use in a method of medical treatment.

16. A pharmaceutical composition comprising the substance of claim 10.

17. Use of a substance of claim 10 for identifying (i) binding partners of the substance or (ii) compounds having the property of binding to Chk1 or Chk2 and inhibiting the phosphorylation of Cdc25A.

18. A method of identifying compounds capable of modulating the interaction of Cdc25A and Chk1 or Chk2, the method comprising:
(a) contacting (i) a substance comprising Cdc25A or a fragment or variant thereof, (ii) a substance comprising Chk1 or Chk2 or a fragment or variant thereof and (iii) a candidate compound, under conditions wherein, in the absence of the candidate compound, said substances interact; and,
(b) determining the interaction between said substances to identify whether the candidate compound modulates the interaction.

19. The method of claim 18, wherein the interaction determined in step (b) is the binding of Cdc25A to Chk1 or Chk2.

20. The method of claim 18, wherein interaction determined in step (b) is the phosphorylation of Cdc25A by Chk1 or Chk2, or the presence or amount of Cdc25A present in a cell based assay.

21. The method of any one of claims 18 to 20, wherein the compound capable of modulating the interaction of Cdc25A and Chk1 or Chk2 is capable of inhibiting the interaction and/or the phosphorylation of Cdc25A by Chk1 or Chk2.

22. The method of any one of claims 18 to 21, wherein the Cdc25A is fusion of GST and a fragment of Cdc25A comprising an amino acid sequence corresponding to the Ser123 of full length Cdc25A.

23. The method of any one of claims 18 to 22, comprising determining the modulation of the interaction of Cdc25A and Chk1 or Chk2 by measuring the phosphorylation of the Cdc25A peptide.

24. The method of claim 23, wherein the phosphorylation of Cdc25A is measured by the incorporation of radioactive phosphate into the Cdc25A peptide.

25. The method of claim 23, wherein the phosphorylation of Cdc25A is determined using an antibody capable of specifically binding to phosphorylated Cdc25A peptide.

26. The method of any one of claims 18 to 25, further comprising testing a candidate compound identified in step (b) to determine whether it is capable of causing G1/S arrest in a population of cells.

27. A method of identifying binding partners of a substance having the property of binding to Chk1 or Chk2 and inhibiting the phosphorylation of Cdc25A by the Chk1 or Chk2, the substance comprising a peptide fragment of between 5 and 30 amino acids having at least 80% sequence identity with a corresponding sequence of Cdc25A, the fragment including serine at a position corresponding to amino acid Ser123 or Ser262 or Ser292 or Ser504 in Cdc25A, the method comprising contacting the substance and a candidate compound and determining whether the candidate compound has the property of binding to the substance.

28. The method of claim 27, further comprising testing the compounds which bind to Cdc25A for activity in inhibiting the phosphorylation of Cdc25A by Chk1 or Chk2.

29. The method of claim 27 or claim 28, further comprising testing said candidate compound to determine whether it is capable of causing G1/S arrest in a population of cells.

30. The method of any one of claims 18 to 29, wherein a plurality of candidate compounds are contacted with said substances.

31. The method of claim 30, wherein the plurality of compounds are present in a compound library.

32. Use of an amino acid motif having between 2 and 30 amino acids from Cdc25A and having a serine at a position corresponding to Ser123 or Ser262 or Ser292 or Ser504 in full length Cdc25A in the design of an compound which is modelled to resemble the three dimensional structure, the steric size, and/or the charge distribution of said amino acid motif, the wherein the compound has the property of binding to Chk1 or Chk2.

33. Use of a substance which is capable of disrupting the interaction of phosphorylated Cdc25A and a F-box protein involved in its degradation for the preparation of a medicament for the treatment of cancer or a hyperproliferative disorder, wherein the inhibition of the interaction inhibits the degradation of the Cdc25A in response to DNA damage.

34. The use of claim 33, wherein the substance comprises:
(a) a peptide fragment of between 5 and 30 amino acids which has at least 80% sequence identity with a corresponding sequence of Cdc25A, the fragment including a serine residue at a position corresponding to amino acid Ser123 or Ser262 or Ser292 or Ser504 in Cdc25A, wherein the serine residue is phosphorylated; or,
(b) a derivative of peptide fragment (a); or
(c) a substance which is peptide fragment (a) or derivative (b) linked to a coupling partner.

35. A method of identifying compounds capable of inhibiting the ubiquitination and degradation of phosphorylated Cdc25A upon binding a F-box protein, the method comprising:
(a) contacting (i) a substance comprising Cdc25A or a fragment or variant thereof, (ii) a F-box protein or a complex including a F-box protein and (iii) a candidate compound, under conditions wherein, in the absence of the candidate compound, the F-box protein targets the Cdc25A for ubiquitination and degradation; and,
(b) determining whether the compound inhibits the interaction of Cdc25A and the F-box protein or the degradation of the Cdc25A.

36. Use of a substance which is:
(a) a peptide fragment of between 5 and 30 amino acids which has at least 80% sequence identity with a corresponding sequence of Cdc25A, the fragment including a serine residue at a position corresponding to amino acid Ser123 or Ser262 or Ser292 or Ser504 in Cdc25A; or,
(b) a peptide fragment of (a) wherein the serine residue is phosphorylated; or,
(c) a derivative of peptide fragment (a) or (b); or
(d) a substance which is peptide fragment (a) or (b) or derivative (c) linked to an immunogenic carrier;
for raising antibodies capable of specifically binding to the Cdc25A.

37. The peptide fragment of claim 10 comprising between 7 and 15 amino acids which has at least 90% sequence identity with a corresponding sequence of Cdc25A.

38. The peptide fragment of claims 10 or 37, consisting of about 11 amino acid residues identical with a corresponding sequence of Cdc25A wherein the serine residue (at one of positions 123, 262, 292 or 504) is substituted with alanine, leucine or a serine analog which can not be phosphorylated.

39. The peptide fragment of claims 10, 37 or 38, wherein the C-terminus is amidated.

40. The peptide fragment of claim 10, 37-39, wherein the fragment is linked to a coupling partner selected from the group consisting of HIV tat peptide residues 49-57, HIV tat peptide residues 49-56, the tat sequence YGRKKRRQRRR, a polyarginine peptide having from 6 to 20 residues, such as R6, and transducing peptide sequences, such as the following peptide sequences: YARKARRQARR, YARAAARQARA, YARAARRAARR, YARAARRAABA, ARRRRRRRRR, and YAAARRRRRRR.

41. The peptide fragment of claims 10, 37-40, wherein the coupling partner is covalently linked to the N-terminus of the fragment.

42. The peptide fragment of claims 10, 37-41, which is selected from the group consisting of YGRKKRRQRRR-LFDSPALCSSS-NH2 (KB8(25A-S262A)), YGRKKRRQRRR-TKRRKAMSGAS-NH2 (KB7(25A-S292A)), YGRKKRRQRRR-KFRTKATRWAG-NH2, YGRKKRRQRRR-LKRSHADSLDH-NH2, YARKARRQARR-LFDSPALCSSS-NH2, YARKARRQARR-TKRRKAMSGAS-NH2, YARKARRQARR-KFRTKLTRWAG-NH2, YARKARRQARR- LKRSHLDSLDH-NH2, YARAARRAARR-LFDSPALCSSS-NH2, YARAARRAARR-TKRRKAMSGAS-NH2, YARAARRAARR-KFRTKLTRWAG-NH2, YARAARRAARR-LKRSHLDSLDH-NH2, YGRKKRRQRRR-LFDSPSLCSSS-NH2, YGRKKRRQRRR-TKRRKSMSGAS-NH2, YGRKKRRQRRR-KFRTKSTRWAG-NH2, YGRKKRRQRRR-LKRSHSDSLDH-NH2, YARKARRQARR-LFDSPSLCSSS-NH2, YARKARRQARR-TKRRKSMSGAS-NH2, YARKARRQARR-KFRTKSTRWAG-NH2, YARKARRQARR-LKRSHSDSLDH-NH2, YARAARRAARR-LFDSPSLCSSS-NH2, YARAARRAARR-TKRRKSMSGAS-NH2, YARAARRAARR-KFRTKSTRWAG-NH2, and YARAARRAARR-LKRSHSDSLDH-NH2.

43. The method of any one of claims 18 to 25 further comprising testing a candidate compound identified in step (b) to determine whether it is capable of causing cell cycle arrest in a population of cells.

44. The method of claim 43, wherein the cell cycle arrest is a G1/S arrest.

45. Use of a substance which is capable of disrupting the interaction of phosphorylated Cdc25A and the proteolytic machinery involved in its degradation for the preparation of a medicament for the treatment of cancer or a hyperproliferative disorder, wherein the inhibition of the interaction inhibits the degradation of the Cdc25A in response to DNA damage.

46. Use according to claim 36 wherein the substance is selected from the group consisting of CGCSPALKRSHSDSLDHDIFQL, CGCSPALKRSHS (H₂PO₃) DSLDHDIFQL, CKEDLKKFRTKSTRWAGEKSKR and CKEDLKKFRTKS (H₂PO₃) TRWAGEKSKR.

47. A method of identification of patients having a functional Cdc25A regulated cell cycle pathway in cancer cells comprising measuring the presence or absence of cell cycling following treatment of the cells with chemotherapy or radiotherapy.

48. The method of claim 47, wherein the chemotherapy includes the administration of a DNA topoisomerase toxin.

49. The method of claim 48, wherein the DNA topoisomerase toxin is an anthracycline, an epipodophyllotoxine or a camptothecin derivative.

50. The method of claim 47, wherein the radiotherapy includes the use of γ-radiation.

51. A diagnostic kit for the identification of patients expressing Cdc25A in cancer cells comprising an antibody against Cdc25A.

52. The diagnostic kit of claim 51, wherein the antibody is raised against a substance of claim 46.

53. A method of screening a patient population by determining the level of Cdc25A expression in cancer cells derived from each patient comprising
(a) providing a sample comprising tissue material or cells from a tumor,
(b) preparation of the sample to obtain an appropriate tissue extract,
(c) optionally further treating the sample extract by one or more purification processes, such as precipitation and SDS-PAGE,
(d) contacting the tissue preparation with an antibody against Cdc25A to produce a Cdc25A primary complex,
(e) contacting the complex with a secondary antibody containing a specific label or reporter group to enable determination of the amount of Cdc25A present; and
(f) comparing the amount of Cdc25A with a standard sample to determine whether it is more or less than the standard amount.

54. The method of claim 53 wherein the level of Cdc25A is determined using the Western blot method or ELISA.

55. A method of screening a patient population by determining the level of Cdc25A expression in cancer. cells derived from each patient comprising measuring the amount of Cdc25A mRNA in the cells using PCR.

56. A method of sensitizing a patient for chemotherapy or radiotherapy comprising administering at least one drug capable of preventing or reducing action of a Cdc25A degradation pathway.

57. The method of claim 56, wherein the drug is a peptide or peptide mimetic.
